SEQUENCE LISTING (1 sequence)

SEQ ID NO: 1 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

(19) United States
(12) Patent Application Publication
(10) Pub. No.: US 2006/0204981 A1
(43) Pub. Date: Sep. 14, 2006

(54) Compositions for modulation of PARP and methods for screening for same

(76) Inventors: Chiang Li, West Roxbury, MA (US); Aijin Wang, Norwood, MA (US); Xiangao Sun, Brookline, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/328,993

(22) Filed: Jan. 9, 2006

Related U.S. Application Data
(60) Provisional application No. 60/642,353, filed on Jan. 7, 2005.

(51) Int. Cl. C12Q 1/68; G01N 33/574; A61K 31/353

(52) U.S. Cl. 435/6; 435/7.23; 514/454

(57) ABSTRACT

The present invention relates a method for screening for a PARP activator. The screening method comprises the step of assessing the PARP-activating effect of a test compound, using cells, cell lysate, or purified PARP. The present invention also provides a method for the treatment of cancers. The treatment method comprises administering to the subject a therapeutically effective amount of a PARP activator.

### CROSS-REFERENCE TO RELATED APPLICATIONS

This application claims the benefit of U.S. Provisional Application No. 60/642,353, filed Jan. 7, 2005, the contents of which are incorporated herein by reference in their entirety.

### BACKGROUND OF THE INVENTION

Poly (ADP-ribose) polymerases (PARP; also known as “poly(ADP-ribose) synthetases”) are a family of nuclear enzymes that use the oxidized form of nicotinamide adenine dinucleotide (“NAD⁺”) as a substrate to synthesize ADP-ribose polymer and transfers the polymer onto other proteins (“poly ADP-ribosylation”). Many proteins can be modified by PARP, such as DNA ligases, DNA and RNA polymerases, endonucleases, histones, topoisomerases and PARP itself. (Nguewa, et al., Mol Pharmacol 64:1007-1014 (2003); Tentori, et al., Pharmacological Research 45:73-85 (2002); Amé, et al, Bioassays 26:882-893 (2004))

18 members have been identified for the PARP family (Amé, et al, Bioassays 26:882-893 (2004)). Among them, PARP-1 and PARP-2 have been shown to be responsive to DNA damage. Their catalytic activity is immediately stimulated by DNA strand breaks. PARP-1, a well-studied PARP, is an enzyme with a molecular mass of 113 kDa (De Murcia et al, BioEssays, 13:455-462 (1991)). PARP-1 is regarded as a dual regulator of cell functions: it is involved either in DNA repair or in cell death. When the DNA damage is moderate, PARP-1 plays a role in the DNA repair. When the DNA injury is massive, however, excessive PARP-1 activation leads to depletion of NAD+/ATP and thereby cell death by necrosis. Indeed, excessive PARP-1 activation and the consequent cell death have been linked to pathogenesis of several diseases, including stroke, myocardial infarction, diabetes, shock, neurodegenerative disorder, allergy, and several other inflammatory processes (Tentori, et al., Pharmacological Research 45:73-85 (2002); Nguewa, et al., Mol Pharmacol 64:1007-1014 (2003)).

PARP-2, having a molecular weight of 62 kDa, has an overlapping role for PARP-1. Knockout of both PARP-1 and PARP-2 genes are lethal to mice, while PARP-1 deficiency by itself is not lethal to mice. (ibid.)

Because of their important roles in DNA repair or in cell death, PARP inhibitors can be used in the treatment of various diseases. On the one hand, PARP inhibitors can be used as adjuvant drugs in cancer therapy, specifically as chemosensitizing and radiosensitizing agents in chemotherapy and radiotherapy. The inhibition of PARP activity suppresses the machinery of DNA repair, of which PARP-1 and PARP-2 are known to be key members. Thus, the suppression of DNA repair increases cell susceptibility of DNA damaging agents and inhibits strand rejoining. The accumulation of the DNA damage in turn leads to cell death by apoptosis.

On the other hand, PARP inhibitors can be used as drugs for the treatment of diseases such as diseases, including stroke, myocardial infarction, diabetes, shock, neurodegenerative disorder, allergy, and several other inflammatory processes. PARP inhibitors can suppress the excessive PARP activation and thereby prevent the cell death caused by the depletion of NAD+/ATP. (ibid.)

β-lapachone is known to be a potent and selective anti-tumor compound. The roles of β-lapachone in the modulation of PARP is unclear yet. One study shows that PARP activity is inhibited by β-lapachone (Villamil S. F., et al. Mol Biochem Parasitol. 115(2):249-56 (2001)), while another study shows that PARP activity is involved in the necrosis of U2-OS cells induced by β-lapachone (Liu T. J., et al. Toxicol Appl Pharmacol. 182(2):116-25 (2002)). Villamil's data of inhibitory role of β-lapachone in PARP activity is inconsistent with Liu's showing of enhanced PARP activity after treatment with β-lapachone.

No single drug or drug combination is curative for advanced metastatic cancer and patients typically succumb to the cancers in several years. Thus, new drugs or combinations that can prolong onset of life-threatening tumors and/or improve quality of life by further reducing tumor-load are very important. There exists a need for the isolation of other anti-proliferative compounds for the treatment of cancer and other hyper-proliferative diseases. Disclosed herein are methods for screening for these compounds, and methods of modulating apoptosis using these compounds.

The references cited herein are not admitted to be prior art to the claimed invention.

### SUMMARY OF THE INVENTION

The present invention relates to a method for screening for a PARP activator. The method comprises the step of assessing the PARP-activating effect of a test compound in cells containing DNA encoding PARP. The PARP can be PARP-1, PARP-2, or both PARP-1 and PARP-2. In an embodiment, the step of assessing the PARP-activating effect in cells comprises exposing the cells to a test compound, measuring the activity of PARP in the cells in the presence and in the absence of the test compound, and comparing the activity of PARP in the presence and in the absence of the test compound. The PARP-activating effect can be determined by an increase in poly(ADP ribose) synthesis.

In an embodiment, the cells used in the screening are cancer cells. The cancer cells can be the cells in a cancer, the cancer cells derived from a cancer, or cultured cancer cells. The cancer can be from a vertebrate, mammal, or human. The examples of the cultured cancer cells include MCF-7 (human breast cancer cells), DLD1 (human colonic cells), SW480 (human colonic cells), and Paca-2 (human pancreatic cancer cells).

The test compound can be a small molecule, and preferably an analog, derivative, or metabolite of β-lapachone.

The present invention also provides a method of for screening for a selective activator of PARP. The method further comprises the step of assessing the PARP-activating effect of the test compound in normal cells containing DNA encoding PARP. In an embodiment, the step of assessing the PARP-activating effect in normal cells comprising exposing the normal cells to a test compound, measuring the activity of PARP in the normal cells in the presence and in the absence of the test compound, and comparing the activity of PARP in the presence and in the absence of the test compound.

The normal cells can be normal cells in a vertebrate, mammal, or human, normal cells derived from a vertebrate, mammal, or human, or cultured normal cells. The examples of the cultured normal cells include MCF-10A (nontransformed breast epithelial cells), NCM460 (normal colonic epithelial cells), PBMC (proliferating peripheral blood mononuclear cells). The method further comprises the step of selecting the test compound that has a higher PARP-activating effect in the cancer cells than in the normal cells.

The present invention further provides a method for screening for a PARP activator using cell lysate. The method comprises the step of assessing the PARP-activating effect of a test compound in the lysate of cells containing DNA encoding PARP. In an embodiment, the cells are cancer cells. The method may further comprises assessing the PARP-activating effect of the test compound in the lysate of normal cells containing DNA encoding PARP, and comparing the PARP-activating effects of the test compound in the cancer cell lysate and the normal cell lysate.

The present invention further provides a method for screening for a PARP activator using PARP. The method comprises contacting PARP with a test compound, measuring the activity of PARP in the presence and in the absence of the test compound, and comparing the activity of PARP in the presence and in the absence of the test compound. In an embodiment, the PARP is PARP-1 or PARP-2. The method may further comprise selecting the test compound that increases the PARP activity. After the compound has been selected, the method may further comprise assessing the PARP-activating effect of the selected compound in cancer cells containing DNA encoding PARP, or the lysate of the cells, assessing the PARP-activating effect of the selected compound in the lysate of normal cells containing DNA encoding PARP, or the lysate, and comparing the PARP-activating effects of the selected compound in the cancer cells or the lysate and the normal cells or the lysate.

The present invention further relates to a method of treating or preventing cancer in a subject. The method comprises comprising increasing PARP activity, preferably selectively increasing PARP activity, in cancer cells of the subject. The method may comprise administering to the subject a therapeutically effective amount of a PARP activator, preferably a selective activator of PARP. The compound can be an analog, derivative, or metabolite of β-lapachone. The subject can be a vertebrate, mammal, or human.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1A is a graph showing the percent survival of HeLa cells in various concentrations of β-lapachone with and without 3-aminobenzamide. FIG. 1B is a graph showing the percent survival of DLD1 cells in various concentrations of β-lapachone with and without 3-aminobenzamide.

FIG. 2 shows a series of light micrographs of Trypan Blue staining of HeLa cells in various concentrations of β-lapachone with and without 3-aminobenzamide.

FIG. 3 shows a series of light micrographs of Trypan Blue staining of DLD1 cells in various concentrations of β-lapachone with and without 3-aminobenzamide.

FIG. 4A shows a fluorescence micrograph of anti-poly(ADP-ribose) antibody on HeLa cells treated with DMSO (control). FIG. 4B shows a fluorescence micrograph of anti-poly(ADP-ribose) antibody on HeLa cells treated with 4 μM β-lapachone for 5 minutes. FIG. 4C shows a fluorescence micrograph of anti-poly(ADP-ribose) antibody on HeLa cells treated with 4 μM β-lapachone for 10 minutes. FIG. 4D shows a fluorescence micrograph of anti-poly(ADP-ribose) antibody on HeLa cells treated with 4 μM β-lapachone for 20 minutes. FIG. 4E shows a fluorescence micrograph of anti-poly(ADP-ribose) antibody on HeLa cells treated with 4 μM β-lapachone for 30 minutes. FIG. 4F shows a fluorescence micrograph of anti-poly(ADP-ribose) antibody on HeLa cells treated with 4 μM β-lapachone for 1 hour. FIG. 4G shows a fluorescence micrograph of anti-poly(ADP-ribose) antibody on HeLa cells treated with 4 μM β-lapachone for 2 hours.

FIG. 5A shows a fluorescence micrograph of anti-poly(ADP-ribose) antibody on HeLa cells treated with DMSO (control). FIG. 5B shows a fluorescence micrograph of anti-poly(ADP-ribose) antibody on HeLa cells treated with 4 μM β-lapachone for 10 minutes. FIG. 5C shows a fluorescence micrograph of anti-poly(ADP-ribose) antibody on HeLa cells treated with 4 μM β-lapachone and 5 mM 3-aminobenzamide for 10 minutes.

FIG. 6A shows a fluorescence micrograph of anti-poly(ADP-ribose) antibody on DLD1 cells treated with DMSO (control). FIG. 6B shows a fluorescence micrograph of anti-poly(ADP-ribose) antibody on DLD1 cells treated with 4 μM β-lapachone for 10 minutes. FIG. 6C shows a fluorescence micrograph of anti-poly(ADP-ribose) antibody on DLD1 cells treated with 4 μM β-lapachone and 5 mM 3-aminobenzamide for 10 minutes.

FIG. 7 is a graph showing the percent of cellular NAD⁺ remaining in DLD1 cells treated with various concentrations of β-lapachone.

FIG. 8 is a graph showing the percent survival of DLD1 cells treated with various concentrations of β-lapachone with and without addition of exogenous NAD⁺.

FIG. 9 is a graph showing the fold activation of PARP activity in various concentrations of β-lapachone in cellular lysate from DLD1 cells.

FIG. 10A shows a Western blot of E2F1 in human colon cancer cell lines (DLD1) which are p53 deficient and trasfected with a tetracycline inducible promoter operably linked to an exogenous E2F1 gene.

FIG. 10B shows flow cytometry data for E2F1 tet-inducible DLD1 cells incubated with tetracycline for 3 and 4 days.

FIG. 10C is a light micrograph of E2F1 tet-inducible DLD1 cells, incubated with tetracycline for 3 and 4 days.

FIG. 10D is a photograph of a colony forming assay using the E2F1 tet-inducible DLD1 cells.

FIG. 10E shows a Western blot of caspase-3 in E2F1 tet-inducible DLD1 cells with tetracycline for various periods of time.

FIG. 10F is a bar graph showing percent apoptosis of E2F1 tet-inducible DLD1 cells when incubated with the pancaspase inhibitor Z-VAD at 50 μM and tetracycline.

FIG. 11A shows an immunoblot of PAR in E2F1 tet-inducible DLD1 cells with varying incubations of tetracycline.

FIG. 11B is a light micrograph of the E2F1 tet-inducible DLD1 cells stained for PAR and DAPI.

FIG. 11C is a bar graph showing the percent apoptosis in the E2F1 tet-inducible DLD1 cells when incubated with 3′-aminobenzamide and tetracycline.

FIG. 12A shows a Western blot of PARP in E2F1 tet-inducible DLD1 cells exposed to PARP-1 siRNA.

FIG. 12B is a series of light micrographs of E2F1 tet-inducible DLD1 cells showing immunolocalization of PAR relative to DAPI staining.

FIG. 12C is a bar graph showing percent apoptosis of E2F1 tet-inducible DLD1 cells when incubated with PARP siRNA and tetracycline.

FIG. 12D shows a Western blot of PARP, E2F1 and actin in tet-inducible DLD1 cells incubated for various periods of time with tetracycline.

FIG. 12E shows RT-PCR of PARP, E2F1 and actin in tet-inducible DLD1 cells.

FIG. 12F shows a Northern blot of PARP, E2F1 and actin in tet-inducible DLD1 cells incubated for various periods of time with tetracycline.

FIG. 13 shows a Western blot of PARP, E2F1 and actin in tet-inducible DLD1 cells incubated with siRNA for E2F1 and PARP1.

FIG. 14A is a series of light micrographs of E2F1 tet-inducible DLD1 cells showing immunolocalization of cytochrome c relative to DAPI staining and PARP-1 activation.

FIG. 14B is a series of light micrographs of E2F1 tet-inducible DLD1 cells showing immunolocalization of AIF relative to DAPI staining and PARP-1 activation.

FIG. 14C is a series of light micrographs of E2F1 tet-inducible DLD1 cells showing immunolocalization of AIF relative to DAPI staining and control siRNA.

FIG. 14D is a series of light micrographs of E2F1 tet-inducible DLD1 cells showing immunolocalization of AIF relative to DAPI staining and PARP-1 siRNA.

FIG. 15 shows a Northern blot of Atm, P73 and Apaf-1 in E2F1 tet-inducible DLD1 cells incubated with tetracycline for various periods of time.

### DETAILED DESCRIPTION OF THE INVENTION

The present invention relates to a method to screen for a PARP activator, and the use of the PARP activator in the prevention and treatment of cancer. In an embodiment, the PARP activator is an analog, derivative, or metabolite of β-lapachone.

### 1. The Method for the Screening of PARP Activator

The present invention provides a method for screening for a PARP activator comprising the step of assessing the PARP-activating effect of a test compound. As used herein, the “PARP-activating effect of a test compound” refers to the capability of a test compound to increase PARP activity. The terms “increase”, “enhance”, “induce” or “promote” are used interchangeably herein. Further, the terms “decrease”, “reduce”, “inhibit” or “prevent” are used interchangeably herein.

The PARP activity may be determined by the measurement of poly(ADP ribose) synthesis. The measurement of PARP activity is known in the art (see, e.g., Brown and Marala, J. of Pharmacol. and Toxicol. Method 47:137-141 (2002); Cheung and Zhang, Analytical Biochemistry 282:24-28 (2000); and Decker et al., Clinical Cancer Research 5:1169-1172 (1999)). The test compound that increases the PARP activity is a PARP activator. Alternatively, the activity of PARP can be measured through monitoring apoptosis in the cells in the presence and in the absence of PARP inhibitor such as 3-aminobenzamide.

The PARP-activating effect of a test compound may be measured by the ratio of the PARP activity in the presence and in the absence of the test compound. In an embodiment, the PARP activity in the presence of the test compound is about 1.5 fold, about 2 fold, about 4 fold, about 10 fold, about 20 fold, about 40 fold, about 100 fold, about 200 fold, about 500 fold, about 1,000 fold, or more than 1,000 fold of the PARP activity in the absence of the test compound.

A PARP activator may increase the PARP activity via various mechanisms. A PARP activator may increase the transcription, post-transcription, translation, or translocation of PARP, or the combination of the above. A PARP activator may interact with PARP directly, or with the modulator(s) of PARP, or with both. In an embodiment, methods of screening PARP activator comprising screening compounds for their ability to increase the activity or expression of PARP.

The PARP-activating effect of a test compound can be assessed using various systems, such as animal model, cultured cells, cell lysate, or isolated PARP, or the combination of the above.

1.1. The Assessing Method Using Cells

In an embodiment, the method for screening for a PARP activator comprises the step of assessing the PARP-activating effect of a test compound in cells. The cells used for the assessment should contain DNA encoding PARP. The step of assessing the PARP-activating effect may comprise exposing the cells to a test compound, measuring the PARP activity in the cells in the presence and in the absence of the test compound, and comparing the PARP activity in the presence and in the absence of the test compound. The test compound that increases the PARP activity may then be selected as a PARP activator.

In another embodiment, the method of screening PARP activator comprising measuring the ability of the test compound to increase the activity or expression of PARP. In one embodiment, this method includes exposing cells expressing PARP with a test compound, and then measuring the expression of PARP. The expression of PARP in the presence of the test compound is then compared to the expression of PARP in the absence of the test compound. If the expression of PARP in the presence of the test compound is more than the expression of PARP in the absence of the test compound, then the test compound is a PARP activator that induces or promotes PARP expression. The expression can be measured by any means known in the art, for example, Western blotting. The expression of PARP in cells not exposed with the test compound can be about 0%, about 1%, about 10%, about 20%, about 50%, or about 75% of the expression in cells exposed with the test compound.

The cells used for the assessment can be cells directly from a eukaryotic organism, preferably vertebrate, more preferably mammal, and further preferably human. Alternatively, the cells used for the assessment can be cultured cells.

The cells used for the assessment are preferably cancer cells. As used herein, “cancer cells” refer to cells that are derived from primary, metastatic, or blood-bome cancers directly from vertebrate, preferably mammal, more preferably human. The cancer cells will in most cases, but not exclusively, be characterized as displaying the so-called “transformed phenotype”, harboring a genetic defect that confers upon said cells unlimited replicative potential, and additionally exhibiting the ability to grow in an anchorage-independent manner in semi-solid tissue culture medium (soft agar, e.g.) and characterized by the ability to form subcutaneous tumors when injected or implanted into immunologically compromised or sub-lethally irradiated rodents or other animal models. The cancer cells can be cells in a cancer from a vertebrate, mammal, or human. The cancer cells can also be cells in a cancer from a chimera animal, or cells derived from a cancer in a chimera animal. One example of the chimera animal is mice with human xenograft tumors (see, e.g., Calabrese et al, J. Natl. Cancer Inst. 96:56-67 (2004). Alternatively, the cancer cells may be cultured cancer cells, propagated indefinitely as adherent monolayers in sterile polystyrene plates. Examples of the cultured cancer cells include MCF-7 (human breast cancer cells), DLD1 (human colonic cells), SW480 (human colonic cells), and Paca-2 (human pancreatic cancer cells).

Preferably, the PARP activator is a selective activator of PARP, which increases PARP activity in cancer cells more than in normal cell. In order to screen for the selective activator of PARP, the method of for screening for a PARP activator may further comprise the step of assessing the PARP-activating effect of the test compound in normal cells, as well as in cancer cells. The step of assessing the PARP-activating effect in normal cells may comprise exposing the normal cells to a test compound, measuring the activity of PARP in the normal cells in the presence and in the absence of the test compound, and comparing the activity of PARP in the presence and in the absence of the test compound. The test compound that increases the PARP activity in the cancer cells more than in the normal cells may then be selected as a selective activator of PARP.

The selectivity of a PARP activator may be measured by the ratio of PARP-activating effects of a test compound in cancer cells and in normal cells. In an embodiment, the PARP-activating effect of a test compound in cancer cells is about 1.5 fold, about 2 fold, about 4 fold, about 10 fold, about 20 fold, about 40 fold, about 100 fold, about 200 fold, about 500 fold, about 1,000 fold, or more than 1,000 fold of the PARP-activating effect of a test compound in cancer cells.

As used herein, “normal cells” refer to cells that have a limited replicative potential relative to cancer cells and that will cease to divide in culture after a finite number of cell divisions. These normal cells will encompass cells that do not exhibit the so-called “transformed phenotype” of cancer cells, will not grow in an anchorage-independent manner in semi-solid tissue culture medium (soft agar, e.g.) and will not form subcutaneous tumors when injected or implanted into immunologically compromised or sub-lethally irradiated rodents or other animal models. The normal cells may be cells directly isolated from tissues of vertebrates, preferably mammal, more preferably human, such as human dermal fibroblasts from skin biopsies, proliferating peripheral blood mononuclear cells (PBMC) isolated from whole blood, or human epithelial cells isolated from normal breast tissue following reduction mammoplasty. The normal cells can be normal cells in a vertebrate, mammal, or human. Alternatively, the normal cells may be cultured cell lines that have been propagated in vitro and have acquired an increased replicative potential (become “immortalized”) without adopting the transformed phenotype, such as MCF-10A (nontransformed breast epithelial cells) and NCM460 (normal colonic epithelial cells).

In the method screening for selective activator of PARP, the cancer cells and normal cells preferably share some major characteristics. For examples, see Li, et al., PNAS 100:2674-2678 (2003).

1.2. The Assessing Method Using Cell Lysate

In another embodiment, the method for screening for a PARP activator comprises the step of assessing the PARP-activating effect of a test compound in lysate of cells. The cells used for the assessment should contain DNA encoding PARP. The step of assessing the PARP-activating effect may comprise exposing the cell lysate to a test compound, measuring the PARP activity in the cell lysate in the presence and in the absence of the test compound, and comparing the PARP activity in the presence and in the absence of the test compound. The compound that increases the PARP activity may then be selected as a PARP activator.

Cell lysates may be generated by various methods known by the skill of the art. For examples see Current protocols in protein science, John E. Coligan et al., Publisher: New York: Wiley 1995-2002 Edition: (v. 1)

The assessing method preferably uses the lysate of cancer cells. The cancer cells are those described in section 1.1.

Similarly, the PARP-activating effects of a test compound in cancer cell lysate and in normal cell lysate may be compared. The test compound that increases the PARP activity in the cancer cell lysate more than in the normal cell lysate may then be selected as a selective activator of PARP. The selectivity of a PARP activator may be determined as in section 1.1.

1.3. The Assessing Method Using PARP

In another embodiment, the method for screening for a PARP activator comprises contacting PARP with a test compound, measuring the activity of PARP in the presence and in the absence of the test compound, and comparing the activity of PARP in the presence and in the absence of the test compound. The test compound that increases the PARP activity may then be selected as a PARP activator.

In an embodiment, the PARP used for assessing the test compound is PARP-1. In a preferred embodiment, the PARP-1 is isolated PARP-1. In one embodiment, the PARP-1 is a human protein, with the amino acid sequence shown in Table 1. Activity is measured by monitoring activity of PARP is measured by monitoring the amount of poly(ADP ribose) groups.

TABLE 1
Amino acid sequence of human PARP-1. (Genbank Acc. No. P09874.)

| | |
|---|---|
| MAESSDKLYRVEYAKSGRASCKKCSESIPKDSLRMAIMVQSPMFDGKVPHWYHFSCFWKVGHS | (SEQ ID NO: 1) |
| IRHPDVEVDGFSELRWDDQQKVKKTAEAGGVTGKGQDGIGSKAEKTLGDFAAEYAKSNRSTCK | |
| GCMEKIEKGQVRLSKKMVDPEKPQLGMIDRWYHPGCFVKNREELGFRPEYSASQLKGFSLLAT | |
| EDKEALKKQLPGVKSEGKRKGDEVDGVDEVAKKKSKKEKDKDSKLEKALKAQNDLIWNIKDEL | |
| KKVCSTNDLKELLIFNKQQVPSGESAILDRVADGMVFGALLPCEECSGQLVFKSDAYYCTGDV | |
| TAWTKCMVKTQTPNRKEWVTPKEFREISYLKKLKVKKQDRIFPPETSASVAATPPPSTASAPA | |
| AVNSSASADKPLSNMKILTLGKLSRNKDEVKAMIEKLGGKLTGTANKASLCISTKKEVEKMNK | |
| KMEEVKEANIRVVSEDFLQDVSASTKSLQELFLAHILSPWGAEVKAEPVEVVAPRGKSGAALS | |
| KKSKGQVKEEGINKSEKRMKLTLKGGAAVDPDSGLEHSAHVLEKGGKVFSATLGLVDIVKGTN | |
| SYYKLQLLEDDKENRYWIFRSWGRVGTVIGSNKLEQMPSKEDAIEHFMKLYEEKTGNAWHSKN | |
| FTKYPKKFYPLEIDYGQDEEAVKKLTVNPGTKSKLPKPVQDLIKMIFDVESMKKAMVEYEIDL | |
| QKMPLGKLSKRQIQAAYSILSEVQQAVSQGSSDSQILDLSNRFYTLIPHDFGMKKPPLLNNAD | |
| SVQAKVEMLDNLLDIEVAYSLLRGGSDDSSKDPIDVNYEKLKTDIKVVDRDSEEAEIIRKYVK | |
| NTHATTHNAYDLEVIDIFKIEREGECQRYKPFKQLHNRRLLWHGSRTTNFAGILSQGLRIAPP | |
| EAPVTGYMFGKGIYFADMVSKSANYCHTSQGDPIGLILLGEVALGNMYELKHASHISKLPKGK | |
| HSVKGLGKTTPDPSANISLDGVDVPLGTGISSGVNDTSLLYNEYIVYDIAQVNLKYLLKLKFN | |
| FKTSLW | |

Once a test compound is selected as a PARP activator, the activator may be further tested for its selectivity in PARP activation. The further test may follow the procedures described above. So the method may further comprise assessing the PARP-activating effect of the activator in cancer cells containing DNA encoding PARP, or the lysate of the cancer cells, assessing the PARP-activating effect of the activator in normal cells containing DNA encoding PARP, or the lysate of the normal cells, and comparing the PARP-activating effects of the activator in the cancer cells or lysate and the normal cells or lysate. The test compound that increases the PARP activity in the cancer cells or lysate more than in the normal cells or lysate may then be selected as a selective activator of PARP. The selectivity of a PARP activator may be determined as in section 1.1.

1.4. The Method for Screening for Cancer Drug Candidate

The present invention also provides a method (also referred to herein as a “screening assay”) for identifying candidate or test compounds or agents that have an enhancing effect on PARP activation or expression and therefore promote cell death of cancer cells. The present invention also includes compounds identified in the screening assays described herein.

In yet another embodiment, the present invention is directed to a method for identifying a potential therapeutic agent for use in the treatment of pre-cancer or cancer, the method comprising providing cells, tissues, or animals; exposing the cells, tissues, or animals to a composition comprising a test compound, wherein the test compound enhances PARP activity or expression; and monitoring the progression of the pre-cancer or cancer; wherein, if the progression of the pre-cancer or cancer is reduced, the candidate compound is identified as a potential therapeutic agent.

In one embodiment, an assay is a cell-based assay in which a cancer cell is exposed to a test compound and the ability of the test compound to enhance activation or expression of PARP directly or indirectly and reduce the progression of pre-cancer or cancer is determined. The cell, for example, can be of mammalian or human origin, and could be a pre-cancer or cancer cell. Determining the ability of the test compound to reduce the progression of pre-cancer or cancer can be accomplished, for example, by monitoring the progression of the pre-cancer or cancer.

The present invention also provides a method for monitoring the effectiveness of treatment of a subject with a test compound or an agent which enhances the activation or expression of PARP directly or indirectly, comprising the steps of (I) obtaining a pre-administration sample from a subject prior to administration of the agent; (ii) detecting the level of expression or activity of PARP in pre-cancer or cancer cells in the preadministration sample; (iii) obtaining one or more post-administration samples from the subject; (iv) detecting the level of expression or activity of PARP pre-cancer or cancer cells in the post-administration samples; (v) comparing the level of expression or activity PARP of the pre-cancer or cancer cells in the pre-administration sample with the pre-cancer or cancer cells in the post administration sample or samples; and (vi) altering the administration of the agent to the subject accordingly.

Suitable in vitro or in vivo assays can be performed to determine the effect of a composition which enhances PARP activity or expression and whether its administration inhibits growth of pre-cancer or cancer cells. In various specific embodiments, in vitro assays may be performed with representative pre-cancer or cancer cells, to determine if a given therapeutic exerts the desired effect upon the cell type(s). Compounds for use in therapy may be tested in suitable animal model systems including, but not limited to rats, mice, cows, monkeys, rabbits, and the like, prior to testing in human subjects. Similarly, for in vivo testing, any of the animal model system known in the art may be used prior to administration to human subjects.

### 2. The Test Compounds

The test compound can be protein, peptide, peptidomimetic, nucleic acid, small molecule, or other drug candidates. In a preferred embodiment, the test compound is a small molecule. A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

In a preferred embodiment, the small molecule used in the present invention is β-lapachone or an analog, derivative, or metabolite thereof. In another embodiment of the invention, the activity of PARP in a cell contacted with the candidate compound is compared to the activity of PARP in a cell contacted with β-lapachone. If the activity of PARP in the presence of the candidate compound is similar to the activity of PARP in the presence of β-lapachone, then the candidate compound induces or promotes PARP activity and induces or promotes apoptosis, and is useful in the modulation of apoptosis in cells and tissues.

The increase in the expression of PARP can also be achieved with other approaches. Specifically, the methods include anti-sense and RNA interference (RNAi), along with methods of heterologously expressing PARP in a cell. Specific siRNAs and antisense nucleotides for the modulation of expression of PARP are also included in the invention.

2.1. The Libraries of the Test Compounds

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, Anticancer Drug Design 12:145 (1997).

Examples of methods for the synthesis of molecular libraries can be found in the art, for example in: DeWitt, et al., PNAS 90:6909 (1993); Erb, et al., PNAS 91:11422 (1994); Zuckermann, et al., J. Med. Chem. 37:2678 (1994); Cho, et al., Science 261:1303 (1993); Carrell, et al., Angew. Chem. Int. Ed. Engl. 33:2059 (1994); Carell, et al., Angew. Chem. Int. Ed. Engl. 33:2061 (1994); and Gallop, et al., J. Med. Chem. 37:1233 (1994).

Libraries of compounds may be presented in solution (e.g., Houghten, Biotechniques 13:412421 (1992)), or on beads (Lam, Nature 354:82-84 (1991)), on chips (Fodor, Nature 364:555-556 (1993)), bacteria (Ladner, U.S. Pat. No. 5,223,409), spores (Ladner, U.S. Pat. No. 5,233,409), plasmids (Cull, et al., PNAS, 89:1865-1869 (1992)) or on phage (Scott and Smith, Science 249:386-390 (1990); Devlin, Science 249:404406 (1990); Cwirla, et al., PNAS 87:6378-6382 (1990); Felici, J. Mol. Biol. 222:301-310 (1991); Ladner, U.S. Pat. No. 5,233,409).

2.2 The Analog, Derivative, or Metabolite of β-Lapachone

As shown in the examples, β-lapachone is a PARP activator. It was proved, for the first time, that the PARP pathway as the main determinant in the apoptosis induced by β-lapachone. Furthermore, It was discovered that β-lapachone directly activates PARP enzyme.

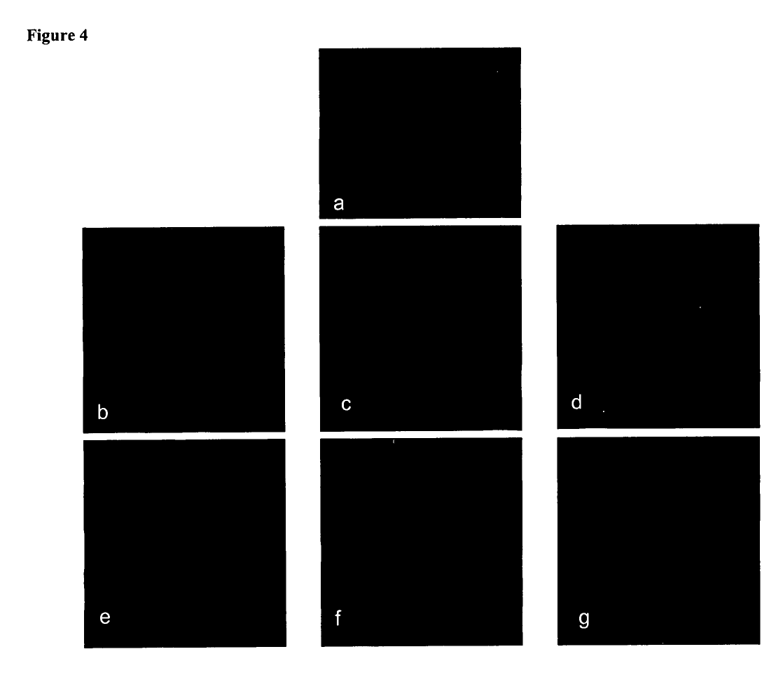

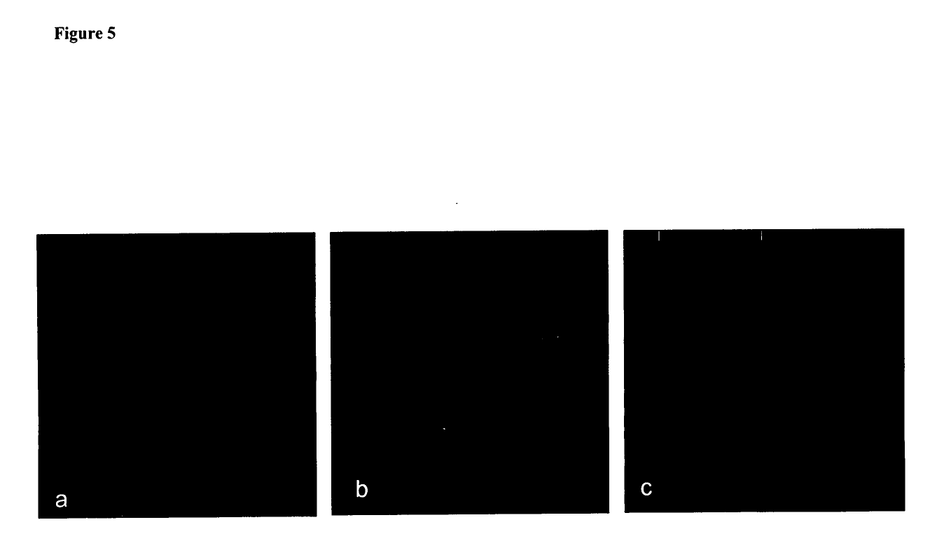

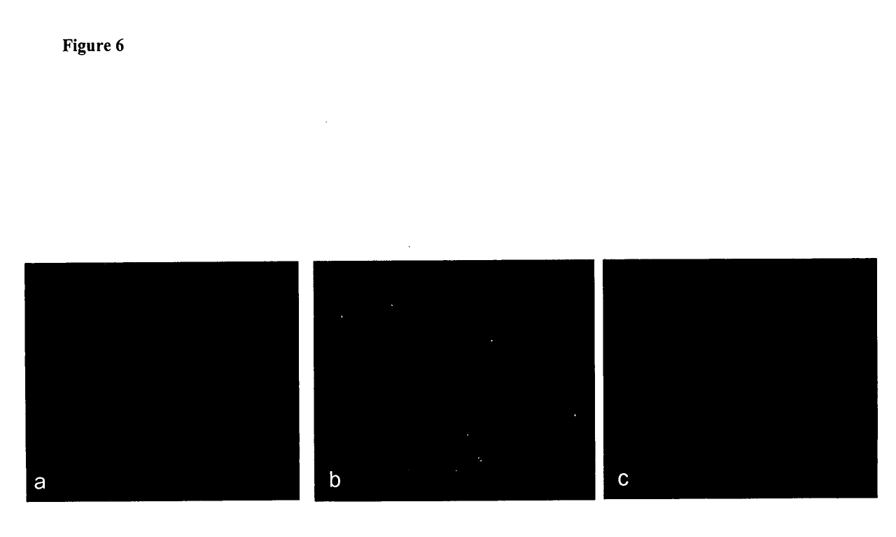

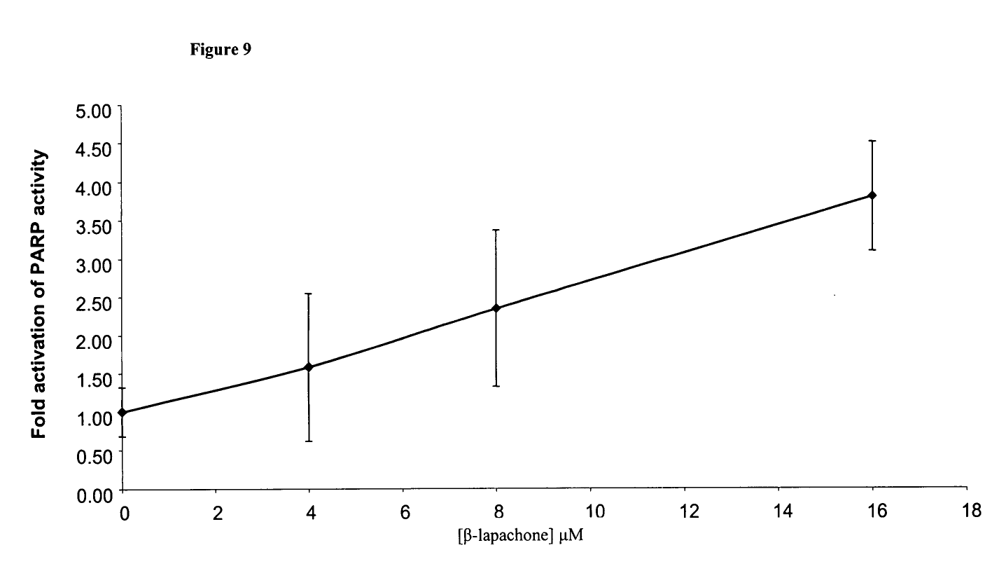

As shown in the examples, β-lapachone enhances PARP activity both in cell lysates (FIG. 9) and in intact cells (FIG. 4). Cells and lysates were treated with β-lapachone, and the amount of PARP product poly(ADP-ribose) was measured. The effect of β-lapachone on enhancement of production of poly(ADP-ribose) by PARP was negated by the addition of known PARP inhibitor 3-aminobenzamide (FIGS. 5 and 6).

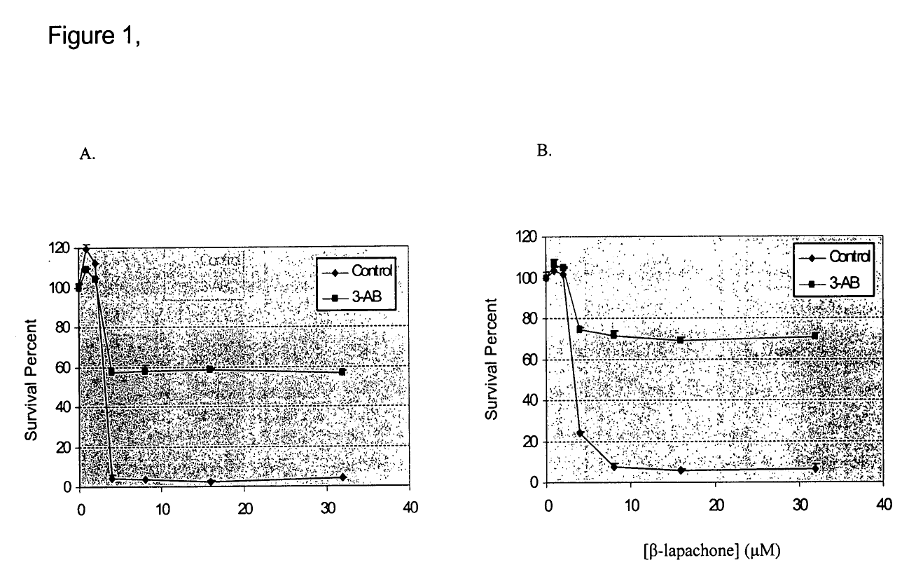

β-lapachone also induces cytotoxicity in HeLa and DLD1 cells (FIGS. 1-3). 3-aminobenzamide blocks the induction of cytotoxicity in HeLa and DLD1 cells (FIGS. 1-3). PARP uses NAD⁺ as a substrate to synthesize poly(ADP) groups. An activation of PARP would cause a depletion of NAD⁺, which would lead to increased cytotoxicity.

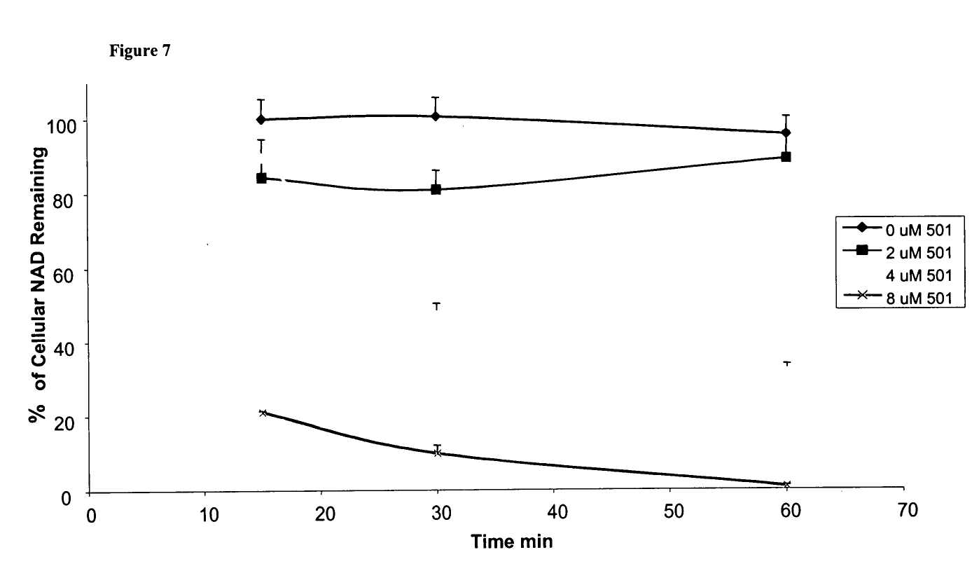

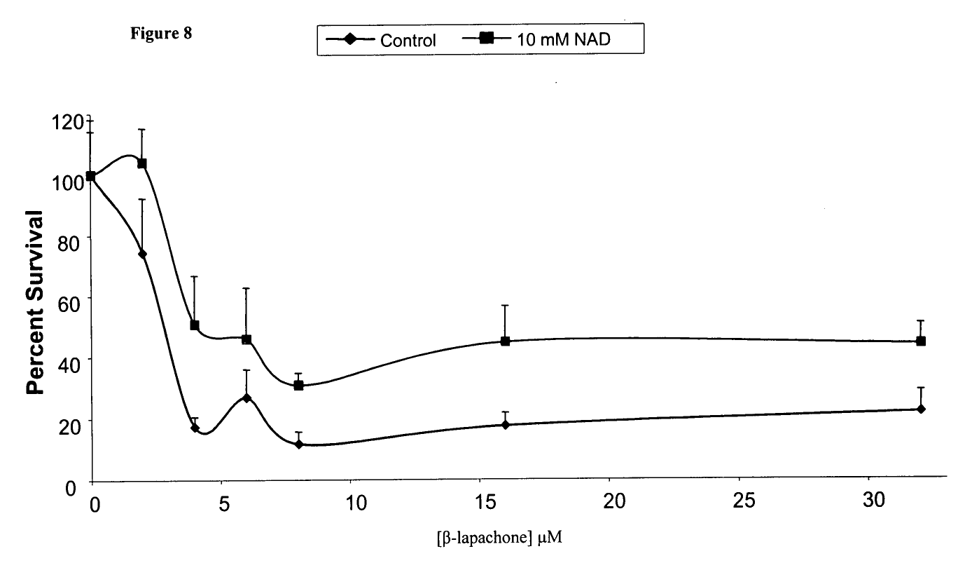

β-lapachone causes the rapid depletion of cellular NAD⁺ levels in cells (FIG. 7). This depletion was inhibited by the addition of 3-aminobenzamide. Increased cytotoxicity associated with β-lapachone administration was reversible through the administration of NAD⁺ (FIG. 8). Similar results were found in MCF7, DLD1, HeLa and SW480 cells. This data shows that β-lapachone directly interacts with PARP, and causes cytotoxicity through enhanced PARP activity.

β-lapachone specifically induces E2F1 in cancer cells, and thereby selectively induces cell death in cancer cells (Li, et al., PNAS 100:2674-2678 (2003)). As shown in the examples, β-lapachone activates PARP activity through E2F1, and inhibition of E2F1 suppresses the PARP activation induced by β-lapachone. Hence, β-lapachone is also a selective activator of PARP, which selectively increases PARP activity in cancer cells.

In one embodiment, the candidate compound is a β-lapachone analog, derivative or metabolite. As further used herein, the phrase “β-lapachone” refers to 3,4-dihydro-2,2-dimethyl-2H-naphtho [1,2-b]pyran-5,6-dione, and has the chemical structure:

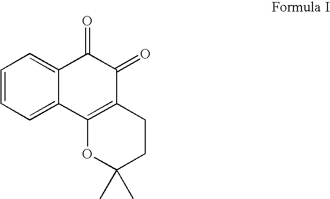

β-lapachone, or analogs, derivatives or metabolites thereof, in accordance with the present invention, can be synthesized as described in U.S. Pat. No. 6,458,974, which is incorporated by reference herein in its entirety. Preferred derivatives and analogs are discussed below.

In another embodiment, analogs of β-lapachone include reduced β-lapachone (Formula Ia, in which R′ and R″ are each hydrogen), as well as derivatives of reduced beta-lapachone (Formula Ia, in which R′ and R″ are each independently hydrogen, lower alkyl, or acyl).

In yet another embodiment, β-lapachone derivatives or analogs, such as lapachol, and 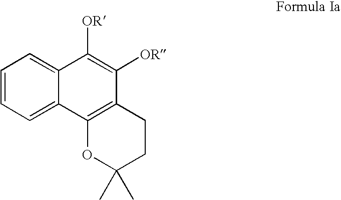
pharmaceutical compositions and formulations thereof are part of the present invention. Such β-lapachone analogs include, without limitation, those recited in PCT International Application PCT/US93/07878 (WO 94/04145), which is incorporated by reference herein in its entirety, and which discloses compounds of the formula: 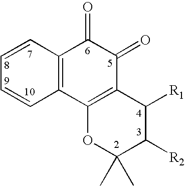
where R₁ and R₂ are each independently hydrogen, substituted and unsubstituted aryl, substituted and unsubstituted alkenyl, substituted and unsubstituted alkyl and substituted or unsubstituted alkoxy. The alkyl groups preferably have from 1 to about 15 carbon atoms, more preferably from 1 to about 10 carbon atoms, still more preferably from 1 to about 6 carbon atoms. The term alkyl unless otherwise modified refers to both cyclic and noncyclic groups, although of course cyclic groups will comprise at least three carbon ring members. Straight or branched chain noncyclic alkyl groups are generally more preferred than cyclic groups. Straight chain alkyl groups are generally more preferred than branched. The alkenyl groups preferably have from 2 to about 15 carbon atoms, more preferably from 2 to about 10 carbon atoms, still more preferably from 2 to 6 carbon atoms. Especially preferred alkenyl groups have 3 carbon atoms (i.e., 1-propenyl or 2-propenyl), with the allyl moiety being particularly preferred. Phenyl and napthyl are generally preferred aryl groups. Alkoxy groups include those alkoxy groups having one or more oxygen linkage and preferably have from 1 to 15 carbon atoms, more preferably from 1 to about 6 carbon atoms. The substituted R₁ and R₂ groups may be substituted at one or more available positions by one or more suitable groups such as, for example, alkyl groups such as alkyl groups having from 1 to 10 carbon atoms or from 1 to 6 carbon atoms, alkenyl groups such as alkenyl groups having from 2 to 10 carbon atoms or 2 to 6 carbon atoms, aryl groups having from six to ten carbon atoms, halogen such as fluoro, chloro and bromo, and N, O and S, including heteroalkyl, e.g., heteroalkyl having one or more hetero atom linkages (and thus including alkoxy, aminoalkyl and thioalkyl) and from 1 to 10 carbon atoms or from 1 to 6 carbon atoms.

Other β-lapachone analogs contemplated in accordance with the present invention include those described in U.S. Pat. No. 6,245,807, which is incorporated by reference herein in its entirety, and which discloses β-lapachone analogs and derivatives having the structure: 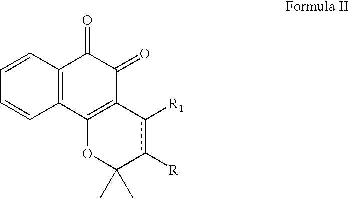
where R and R₁ are each independently selected from hydrogen, hydroxy, sulfhydryl, halogen, substituted alkyl, unsubstituted alkyl, substituted alkenyl, unsubstituted alkenyl, substituted aryl, unsubstituted aryl, substituted alkoxy, unsubstituted alkoxy, and salts thereof, where the dotted double bond between the ring carbons represents an optional ring double bond.

Additional β-lapachone analogs and derivatives are recited in PCT International Application PCT/US00/10169 (WO00/61142), which is incorporated by reference herein in its entirety, and which discloses compounds of the structure:

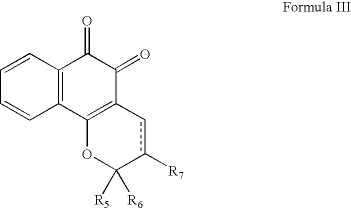

where R₅ and R₆ may be independently selected from hydroxy, C₁-C₆ alkyl, C₁-C₆ alkenyl, C₁-C₆ alkoxy, C₁-C₆alkoxycarbonyl, —(CH₂)ₙ-phenyl; and R₇ is hydrogen, hydroxyl, C₁-C₆ alkyl, C₁-C₆alkenyl, C₁-C₆ alkoxy, C₁-C₆alkoxycarbonyl, —(CH₂)ₙ—amino, —(CH₂)ₙ-aryl, —(CH₂)ₙ-heteroaryl, —(CH₂)ₙ-heterocycle, or —(CH₂)ₙ-phenyl, wherein n is an integer from 0 to 10.

Other β-lapachone analogs and derivatives are disclosed in U.S. Pat. No. 5,763,625, U.S. Pat. No. 5,824,700, and U.S. Pat. No. 5,969,163, as well is in scientific journal articles, such as Sabba et al., J Med Chem 27:990-994 (1984), which discloses β-lapachone with substitutions at one or more of the following positions: 2-, 8- and/or 9-positions. See also Portela et al., Biochem Pharm 51:275-283 (1996) (substituents at the 2- and 9-positions); Maruyama et al., Chem Lett 847-850 (1977); Sun et al., Tetrahedron Lett 39:8221-8224 (1998); Goncalves et al., Molecular and Biochemical Parasitology 1:167-176 (1998) (substituents at the 2- and 3-positions); Gupta et al., Indian Journal of Chemistry 16B: 35-37 (1978); Gupta et al., Curr Sci 46:337 (1977) (substituents at the 3- and 4-positions); DiChenna et al., J Med Chem 44: 2486-2489 (2001) (monoarylamino derivatives). Each of the above-mentioned references are incorporated by reference herein in their entirety.

More preferably, β-lapachone analogs and derivatives contemplated by the present application are intended to encompass compounds having the general formula II and III: 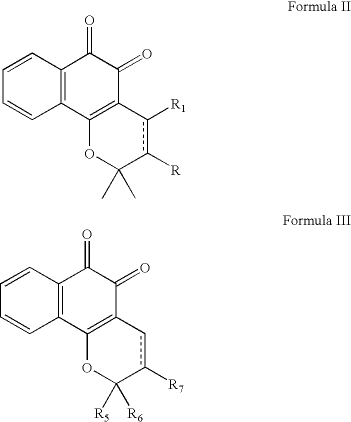
where the dotted double bond between the ring carbons represents an optional ring double bond and where R and R₁ are each independently selected from hydrogen, hydroxy, sulfhydryl, halogen, substituted alkyl, unsubstituted alkyl, substituted alkenyl, unsubstituted alkenyl, substituted aryl, unsubstituted aryl, substituted alkoxy, unsubstituted alkoxy, and salts thereof. The alkyl groups preferably have from 1 to about 15 carbon atoms, more preferably from 1 to about 10 carbon atoms, still more preferably from 1 to about 6 carbon atoms. The term alkyl refers to both cyclic and noncyclic groups. Straight or branched chain noncyclic alkyl groups are generally more preferred than cyclic groups. Straight chain alkyl groups are generally more preferred than branched. The alkenyl groups preferably have from 2 to about 15 carbon atoms, more preferably from 2 to about 10 carbon atoms, still more preferably from 2 to 6 carbon atoms. Especially preferred alkenyl groups have 3 carbon atoms (i.e., 1-propenyl or 2-propenyl), with the allyl moiety being particularly preferred. Phenyl and napthyl are generally preferred aryl groups. Alkoxy groups include those alkoxy groups having one or more oxygen linkage and preferably have from 1 to 15 carbon atoms, more preferably from 1 to about 6 carbon atoms. The substituted R and R₁ groups may be substituted at one or more available positions by one or more suitable groups such as, for example, alkyl groups having from 1 to 10 carbon atoms or from 1 to 6 carbon atoms, alkenyl groups having from 2 to 10 carbon atoms or 2 to 6 carbon atoms, aryl groups having from six to ten carbon atoms, halogen such as fluoro, chloro and bromo, and N, O and S, including heteroalkyl, e.g., heteroalkyl having one or more hetero atom linkages (and thus including alkoxy, aminoalkyl and thioalkyl) and from 1 to 10 carbon atoms or from 1 to 6 carbon atoms; and where R₅ and R₆ may be independently selected from hydroxy, C₁-C₆ alkyl, C₁-C₆ alkenyl, C₁-C₆ alkoxy, C₁-C₆ alkoxycarbonyl, —(CH₂)ₙ-aryl, —(CH₂)ₙ-heteroaryl, —(CH₂)ₙ-heterocycle, or —(CH₂)ₙ-phenyl; and R₇ is hydrogen, hydroxyl, C₁-C₆ alkyl, C₁-C₆alkenyl, C₁-C₆alkoxy, C₁-C₆alkoxycarbonyl, —(CH₂)ₙ-amino, —(CH₂)ₙ-aryl, —(CH₂)ₙ-heteroaryl, —(CH₂)ₙ-heterocycle, or —(CH₂)ₙ-phenyl, wherein n is an integer from 0 to 10.

Preferred β-lapachone analogs and derivatives also contemplated by the present invention include compounds of the following general formula IV: 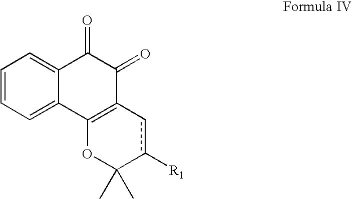
where R₁ is (CH₂)ₙ—R₂, where n is an integer from 0-10 and R₂ is hydrogen, an alkyl, an aryl, a heteroaromatic, a heterocyclic, an aliphatic, an alkoxy, an allyloxy, a hydroxyl, an amine, a thiol, an amide, or a halogen.

Analogs and derivatives also contemplated by the present invention include 4-acetoxy-β-lapachone, 4-acetoxy-3-bromo-β-lapachone, 4-keto-β-lapachone, 7-hydroxy-β-lapachone, 7-methoxy-β-lapachone, 8-hydroxy-β-lapachone, 8-methoxy-β-lapachone, 8-chloro-β-lapachone, 9-chloro-β-lapachone, 8-methyl-β-lapachone and 8,9-dimethoxy-β-lapachone.

Other β-lapachone analogs and derivatives also contemplated by the present invention include compounds of the following general formula V:

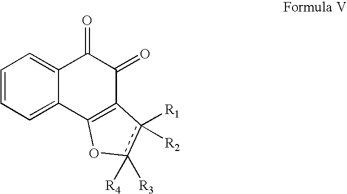

where R₁-R₄ are each, independently, selected from the group consisting of H, C₁-C₆ alkyl, C₁-C₆ alkenyl, C₁-C₆ alkoxy, C₁-C₆ alkoxycarbonyl, —(CH₂)ₙ-aryl, —(CH₂)ₙ-heteroaryl, —(CH₂)ₙ-heterocycle, or —(CH₂)ₙ-phenyl; or R₁ and R₂ combined are a single substituent selected from the above group, and R₃ and R₄ combined are a single substituent selected from the above groups, in which case—is a double bond.

Preferred β-lapachone analogs and derivatives also contemplated by this invention include dunnione and 2-ethyl-6-hydroxynaphtho[2,3-b]-furan-4,5-dione.

Preferred β-lapachone analogs and derivatives also contemplated by the present invention include compounds of the following general formula VI: 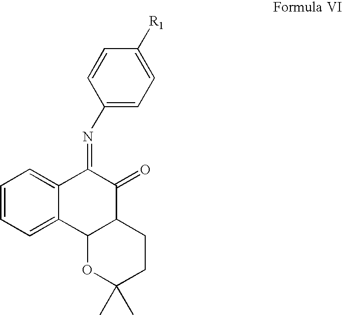
where R₁ is selected from H, CH₃, OCH₃ and NO₂.

Additional preferred β-lapachone analogs useful in the methods and kits of the present invention are recited in PCT International Application PCT/US03/37219 (WO2004/045557), which is incorporated by reference herein in its entirety, and which discloses compounds represented by Formula VII: 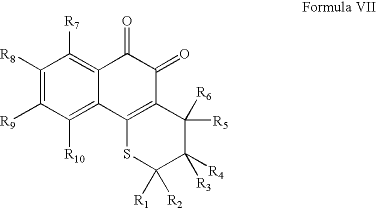
or pharmaceutically acceptable salts thereof, or a regioisomeric mixture thereof, wherein R1-R6 are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R₁ or R₂ and one of R₃ or R₄; or one of R₃ or R₄ and one of R₅ or R₆ form a fused ring, wherein the ring has 4-8 ring members; R₇-R10 are each, independently, hydrogen, hydroxyl, halogen, substituted or unsubstituted alkyl, substituted or unsubstituted alkoxy, nitro, cyano or amide; and n is an integer from 0 to 10.

In a preferred embodiment, R₁ and R₂ are alkyl, R₃-R₆ are, independently, H, OH, halogen, alkyl, alkoxy, substituted or unsubstituted acyl, substituted alkenyl or substituted alkyl carbonyl, and R₇-R₁₀ are hydrogen. In another preferred embodiment, R₁ and R₂ are each methyl and R₃-R₁₀ are each hydrogen. In another preferred embodiment, R₁-R₄ are each hydrogen, R₅ and R₆ are each methyl and R₇-R10 are each hydrogen.

Additional preferred β-lapachone analogs useful in the methods and kits of the present invention are recited in PCT International Application PCT/US03/37219 (WO2004/045557), which is incorporated by reference herein in its entirety, and which discloses compounds represented by Formula VIII:

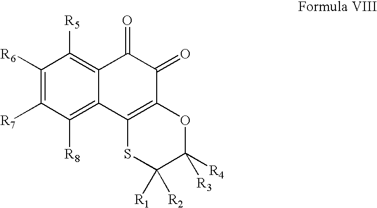

or pharmaceutically acceptable salts thereof, or a regioisomeric mixture thereof, wherein R₁-R₄ are each, independently, selected from the group consisting of H, OH, substituted and unsubstituted C₁-C₆ alkyl, substituted and unsubstituted C₁-C₆ alkenyl, substituted and unsubstituted C₁-C₆ alkoxy, substituted and unsubstituted C₁-C₆ alkoxycarbonyl, substituted and unsubstituted C₁-C₆ acyl, —(CH₂)ₙ-amino, —(CH₂)ₙ-aryl, —(CH₂)ₙ-heterocycle, and —(CH₂)ₙ-phenyl; or one of R₁ or R₂ and one of R₃ or R₄ form a fused ring, wherein the ring has 4-8 ring members; R₅-R₈ are each, independently, hydrogen, hydroxyl, halogen, substituted or unsubstituted alkyl, substituted or unsubstituted alkoxy, nitro, cyano or amide; and n is an integer from 0 to 10. In certain embodiments of Formula VIII, R₁, R₂, R₃, R₄, R₅, R₆, R₇ and R₈ are not each simultaneously H.

### 3. Methods for Diagnosing and Treating Mammalian Pre-Cancer, Cancer or Hyperproliferation Disorders

The PARP activators provided by the present invention can be used as new drugs that kill cancer cells by increasing the activity of PARP. The anticancer drugs promote cell death in cells of a PARP related disorder, such as, pre-cancer or cancer cells, hyperproliferative cells or cells associated with DNA damage.

Various cancers to be treated include but are not limited to lung cancer, colorectal cancer, breast cancer, pancreatic cancer, ovarian cancer, prostate cancer, renal carcinoma, hepatoma, brain cancer, melanoma, multiple myeloma, hematologic tumor, and lymphoid tumor. Hyperproliferative disorders refer to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration. Hyperproliferative diseases/disorders to be treated include but are not limited to epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses and the like. The PARP related disorder can be a DNA repair disorder, including but not limited to, Ataxia-Talangiectasia, premature aging syndrome, Li-Fraumeini syndrome and premalignant conditions, such as BRCA families. The compositions of the present invention may also be useful for pre-cancer, clinical conditions that bear increased risk of progression into cancer.

The present invention is also broadly drawn to the use of the candidate compounds to modulate cell death of cancer cells. Modulation of apoptosis using these test compounds can occur in vitro, in vivo, or ex vivo. Modulation can occur in cancer cells, cell lines, and primary cells.

Another embodiment of the present invention is a method for preventing or inhibiting growth of pre-cancer or cancer cells, the method comprising administering to the cells a composition, which enhances PARP activity or expression in the cell in an amount sufficient to inhibit growth of pre-cancer or cancer cells. The method can be carried out on mammalian cells, including human cells, and can be carried out in vitro or in vivo.

Another embodiment of the present invention is a method for diagnosing and treating mammalian pre-cancer or cancer in a subject, the method comprising obtaining pre-cancer or cancer cells from the subject; testing the pre-cancer or cancer cells from the subject for the presence of PARP; and administering to the subject a composition which enhances PARP activity or expression, in an amount sufficient to inhibit growth of pre-cancer or cancer cells. The composition used in this method can be a β-lapachone analog, derivative, or metabolite thereof, or an mRNA, which increases the expression of PARP.

Another embodiment of the present invention is a method for treating pre-cancer or cancer in a mammalian subject, the method comprising administering to the mammal a composition which enhances PARP activity or expression and monitoring the mammal to determine the state of the pre-cancer or cancer, wherein the composition is administered in an amount sufficient to inhibit the growth of pre-cancer or cancer cells. The composition used in this method can be a β-lapachone analog, derivative, or metabolite thereof, or an mRNA, which increases the expression of PARP.

Another embodiment of the present invention is a method for diagnosing patients who would be receptive to treatment with a composition, which, when administered to mammalian subjects with pre-cancer or cancer, selectively enhances activation or expression of PARP and results in regression of tumor cell growth in the mammalian subjects. The method comprises obtaining cells from the patient; testing the cells for the presence of either PARP or for the presence of PARP activity; wherein the presence in the cells of either PARP or for the presence of PARP activity indicates a patient who would be receptive to treatment.

In this embodiment, compounds such as β-lapachone analogs, derivatives or metabolites thereof could be used to diagnose cancer. Cells isolated from subjects could be cultured in the presence or absence of β-lapachone analogs, derivatives or metabolites thereof. Cells that have their growth rates inhibited in the β-lapachone analogs, derivatives or metabolites thereof treated cells relative to control would be pre-cancer or cancer cells. The subject could then be diagnosed with pre-cancer or cancer.

The PARP activators of the present invention may help the development of drugs that inhibit apoptosis during various forms of tissue injury such as ischemia, reperfusion injury, mechanical injury, inflammation or immunological damage.

3.1 The Compositions for the Test Compounds and PARP Activators

As discussed above, in one aspect, the present invention provides a composition, which, when administered to mammalian subjects with pre-cancer, cancer or a hyper-proliferative disorder, selectively enhances PARP activity and results in regression of cell growth in mammalian cells and subjects. The composition can also be in the form of a pharmaceutical composition or in a kit.

The compositions of the present invention can be incorporated into pharmaceutical compositions suitable for administration. Such compositions typically comprise the substance that enhances PARP activity or expression and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the substance that enhances PARP activity or expression) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### EXAMPLES

Examples are provided below to further illustrate different features of the present invention. The examples also illustrate useful methodology for practicing the invention. These examples do not limit the claimed invention.

### Example 1

### PARP Screening

1. Cell Death Assays

Cell death was determined by the 3-(4,5-dimethylthiazol-2-yl)-2,5-diphenyl tetrazolium bromide (MTT) assay or by trypan blue exclusion, as indicated. Briefly, HeLa and DLD1 cells were plated in a 96-well plate at 10,000 cells per well, cultured for 24 h in complete growth medium, then treated with various concentrations of β-lapachone for 4 h MTT was added to a final concentration of 0.5 mg/ml, and incubated for 1 hr, followed by assessment of cell viability using a microplate reader at 570 nm.

For the trypan blue exclusion assay, HeLa and DLD1 cells were plated in 6-well plate and treated in the same way. They were harvested, and trypan blue dye solution was added to the cell suspension. Total cell counts and viable cell numbers were determined with a hemacytometer. For the PARP inhibition study, cells were pre-teated for 1 h with the PARP inhibitor 3-aminobenzamide (3-AB, 5 mM), and then co-treated with inhibitor and β-lapachone for a further 4 h, followed by MTT assay or trypan blue staining.

For NAD⁺ supplementation experiment, MCF7 cells were plated in 96-well plates at a density of 10,000 cells per well. Sixteen to eighteen hours later cells were pre-treated with 5 μM 3-aminobenzamide, 10 mM NAD+, or vehicle control (all treatments in formulated in growth media: DMEM with 10% fetal bovine serum) for 1 hour at 37° C. Following this incubation, cells (under each pre-incubation treatment) were treated with β-lapachone at indicated concentrations for 4 hours at 37° C. An MTT assay was then performed as described above.

2. Immunofluorescence Analysis

HeLa and DLD1 cells were grown on coverslips for PARP activity experiments. Cells treated with 4 uM β-lapachone at different time points and fixed with methanol acetone (70/30, v/v) for 10 min at −20° C. Coverslips were air dried and rehydrated in PBS at room temperature for 10 min. Samples were than incubated in blocking buffer (PBS, 5% FBS) for 10 minutes at room temperature in a humid chamber. Cells were incubated overnight at 4° C. with monoclonal anti-poly(ADP-ribose) antibody (I OH, 1:100 dilution). After washing, the cells were incubated for 1 hr at room temperature with a 1:500 dilution of FITC conjugated anti-mouse antibody. For the PARP inhibition study, cells pre-teated for 1 h with the PARP inhibitor 3-aminobenzamide (3-AB, 5 mM), and then co-treated with inhibitor and β-lapachone for a further 10 minutes followed by immunofluorescence staining. Immunofluorescence was evaluated using an immunofluorescence microscope equipped with a CCD camera.

3. NAD Depletion Assay

HeLa cells were plated at 6×10⁵ cells per well (35 mm, 6-well dishes). Eighteen hours after plating, cells were treated with β-lapachone (0, 2, 4 or 8 μM) in growth media (DMEM; 10% FBS) for the time periods indicated (15, 30 and 60 minutes). Following drug treatment cells were washed 2× with PBS. Cells were subsequently lysed in 200 μL NAD lysis buffer (61 mM glycyl-glycine, pH 7.4, 0.1% Triton-X-100). Cellular lysates were clarified by centrifugation at 16,000 g for 10 minutes. Aliquots of clarified lysates were transferred to 96-well plates (25 μL samples in triplicate) for to determine NAD⁺ concentrations.

Determination of lysate NAD levels was performed using a modification of the NAD recycling assay described by Ying et al. (PNAS, 98(21):12227-32 (2001)). Briefly, the NAD reaction mixture consisted of 0.1 mM 3-[4,5-dimethylthiazol-2-yl]-2,5-diphenyl-tetrazolium bromide (MTT), 0.9 mM phenazine methosulfate, 13 units/ml alcohol dehydrogenase (from yeast extract, Sigma), 100 mM nicotinamide, and 5.7% ethanol in 61 mM Gly-Gly buffer (pH 7.4). To each aliquot of cellular lysate, 200 μL of reaction mixture was added. Absorbance readings (560 nm) were taken every minute for five minutes. Results were calibrated with NAD⁺ standards and were subsequently normalized to protein concentration as determined using the Bio-Rad protein Assay. Finally, this data was normalized to control treated cells (0 μM β-lapachone at 15 minutes) in order to plot percent cellular NAD⁺ levels remaining.

4. PARP Activity Assay

A cell extract was generated by scraping a 15 cm (˜80% confluent) dish of MCF7 cells into 1.5 ml of PARP buffer (50 mM Tris pH 8.0, 25 mM MgCl₂) containing protease inhibitors. The cell suspension was sonicated at 30% amplitude with 3×10 second bursts on ice. The cell lysate was subsequently clarified by centrifugation at 16,000 g for 10 minutes at 4° C. Any remaining insoluble material was removed by passing the clarified lysate through a 5 ml syringe containing a cotton pad-filter. The protein concentration of extracts was typically between 0.6 and 1.0 μg/ml.

PARP in vitro activity was determined using a modification of the PARP in vitro assay from TREVIGEN™. Reactions were comprised of the following: ˜60 μg of cellular protein, 100 μM NAD, 10 μg of histone H1, β-lapachone (at various concentrations) or DMSO control, 25 mM MgCl₂, 50 mM Tris-Cl pH 8.0. Reactions were incubated for 10 minutes at room temperature and terminated by the addition of 900 μL of cold 25% trichloroacetic acid. Terminated reactions were incubated on ice for 10 minutes. TCA-precipitated proteins were isolated by passing reaction over glass-fiber filters under vacuum. Filters were subsequently washed three times with 5 ml of 5% TCA followed by 2 washes with cold ethanol. Filters were dried and transferred to scintillation cocktail. The amount of ³²P labeled NAD incorporated into polyribosylated proteins was then determined by liquid scintillation measurement.

### Example 2

### β-Lapachone Induction of Cell Death is Inhibited by the PARP Inhibitor 3-Aminobenzamide

MTT assays showed that β-lapachone-induced cell death is blocked by PARP inhibitor 3-aminobenzamide (3-AB). HeLa and DLD1 cells were plated in 96-well plates at 10,000 cells per well, cultured for 24 h in complete growth medium, pretreated with PARP inhibitor 3-AB (SM) or equal volume of DMSO for 1 h, and then exposed to β-lapachone at various concentrations for a further 4 h, followed by MTT assay.

As shown in FIG. 1, HeLa cell survival percentage rises from approximately 5% to 60% in HeLa cells, and to approximately 75% in DLD1 cells.

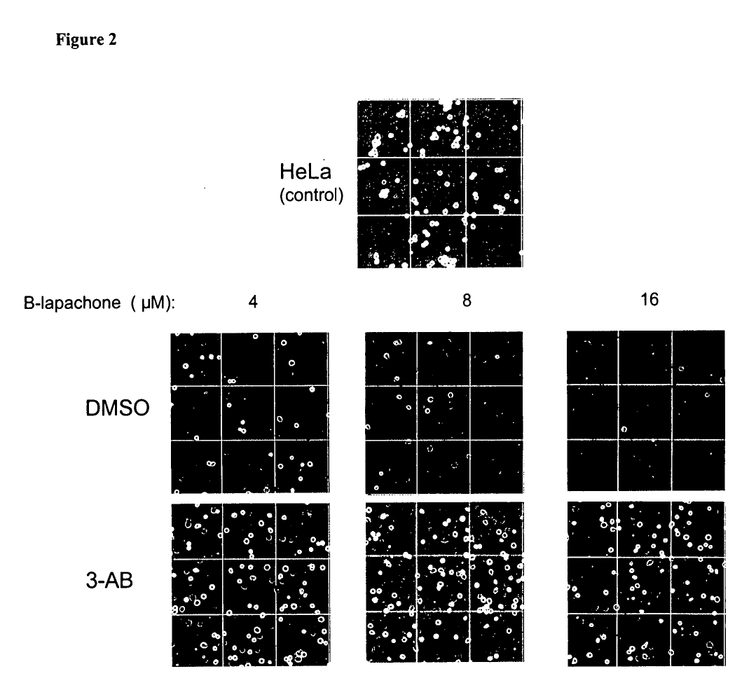

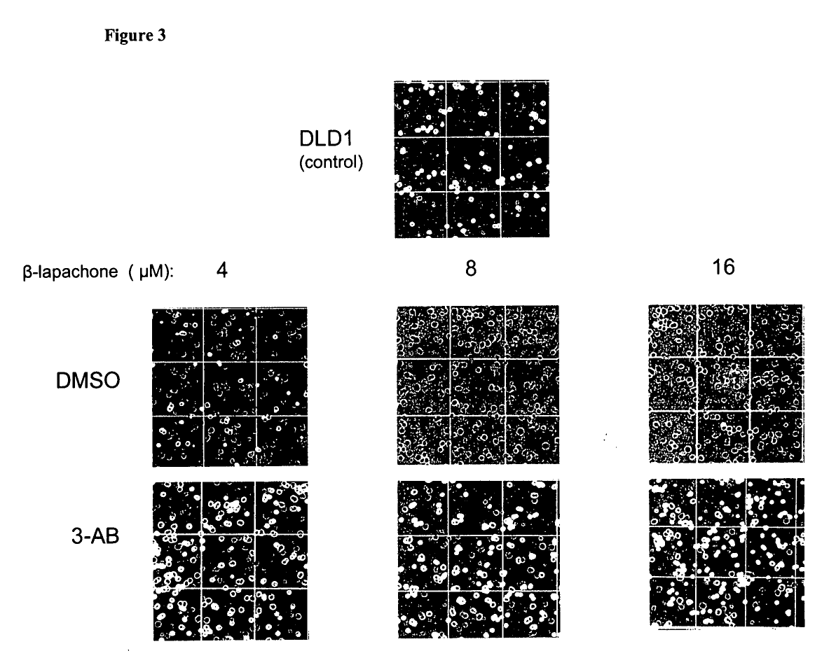

Similar results were shown using Trypan Blue staining in HeLa cells (FIG. 2) and DLD1 cells (FIG. 3).

### Example 3

### β-Lapachone Induces Rapid Cellular Activation of PARP, which is Blocked by 3-Aminobenzamide

β-lapachone induces rapid activation of PARP in HeLa cells. HeLa cells were grown on coverslips for 24 h, then treated with 4 μM β-lapachone at different time points and fixed with methanol acetone (70/30, v/v) for 10 min. Samples were incubated in blocking buffer (5% FBS in PBS) for 10 minutes at room temperature in a humid chamber. Cells were incubated overnight at 4° C. with monoclonal anti-poly(ADP-ribose) antibody (10H 1:100). After washing, the cells were incubated for 1 h at room temperature with FITC conjugated anti-mouse antibody (1:1,000). Immunofluorescence was evaluated using an immunofluorescence microscope equipped with a CCD camera.

As shown in FIG. 4, β-lapachone increases fluorescence due to the presence of monoclonal anti-poly(ADP-ribose) antibody bound to the product of PARP, showing a β-lapachone induced activation of PARP.

β-lapachone induced activation of PARP in HeLa cells is blocked by 3-AB. HeLa cells were grown on coverslips for 24 h, pretreated with 5 mM PARP inhibitor 3-AB or DMSO for 1 h, then exposed to 4 μM β-lapachone for 10 minutes, followed by immunofluorescence staining as described above.

As shown in FIG. 5, β-lapachone induced activation of PARP in HeLa cells, as was also shown in FIG. 4, is inhibited by 3-aminobenamide, verifying that the PARP product, poly(ADP ribose) is indeed generated by PARP and its accumulation is caused by β-lapachone activation of PARP activity.

Similar results were shown using the methods described above for FIG. 5 in DLD1 cells. These results are shown in FIG. 6.

### Example 4

### β-Lapachone Induces NAD⁺ Depletion in Cells, and Reconstitution of NAD⁺ to these Cells Decreases β-Lapachone Induced Cytotoxicity

As shown in FIG. 7, β-lapachone induces a rapid depletion of cellular NAD⁺ levels. Within 15 minutes, the 8 μM β-lapachone treatment reduced NAD⁺ levels to ˜20% of control. By 30 minutes the β-lapachone concentrations in excess of the IC₅₀ (˜2.8 μM) reduced cellular NAD⁺ levels to less than 50% of control. The rapid kinetics of β-lapachone-mediated NAD⁺ depletion implies that this phenomenon is due to an enzymatic process. Furthermore, this process is inhibited by the compound 3-aminobenzamide (data not shown). Together these data suggest that a PARP activity is involved in β-lapachone-induced NAD⁺ depletion.

As shown in FIG. 8, addition of exogenous NAD+ to cell growth media can reduce the cytotoxicitiy of β-lapachone treatment (IC₅₀ values of ˜2.7 and ˜6.6 μM for control versus NAD+supplementation respectively in MCF7 cells). The ability of exogenous NAD⁺ to protect cells from β-lapachone induced cytotoxicity is likely limited by cellular uptake of this charged molecule. Similar results were observed in experiments using DLD1, HeLa and SW480 cells. These results taken together with those in FIG. 7 suggest that the depletion of cellular NAD+levels is a contributing factor to β-lapachone induced cytotoxicity.

### Example 5

### β-Lapachone Induces Activation of PARP in Cell Lysates

FIG. 9 shows that the addition of β-lapachone to cellular lysate induces enhancement of PARP activity above the basal level (0 μM β-lapachone control). Furthermore, this induction of PARP activation by β-lapachone was dependent on β-lapachone concentration (dose-dependent). This observation is significant in that it shows that a PARP family member is a direct target of β-lapachone.

### Example 6

### Induction of E2F1 Expression Promotes Apoptosis in DLD1 and SW480 Cells

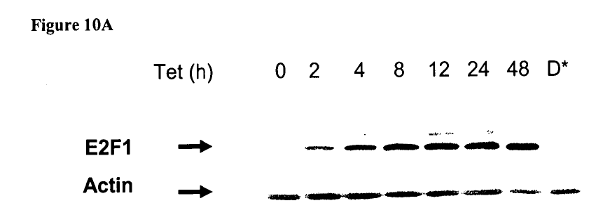

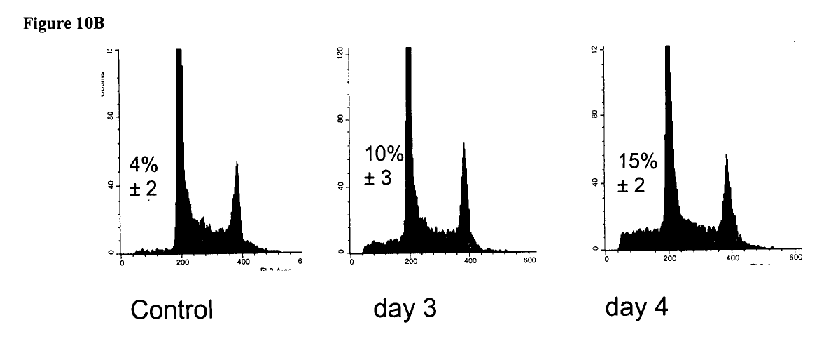

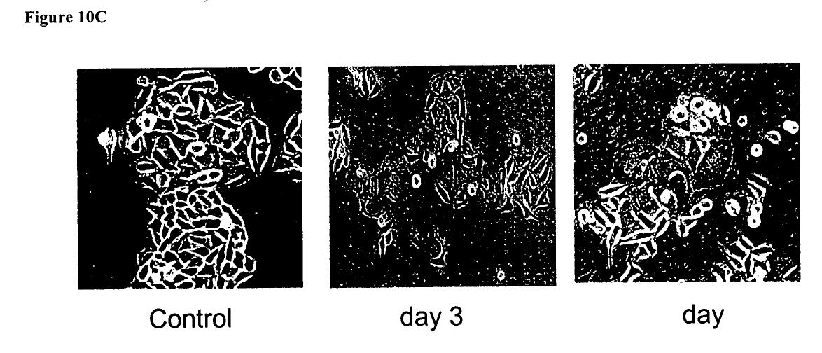

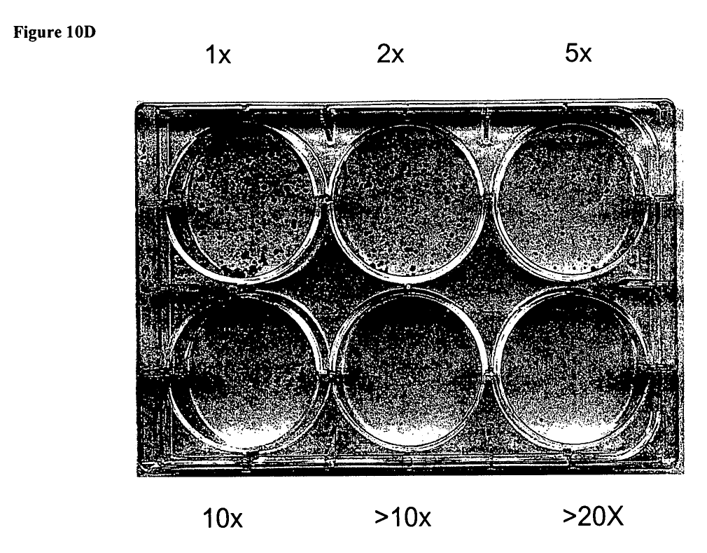

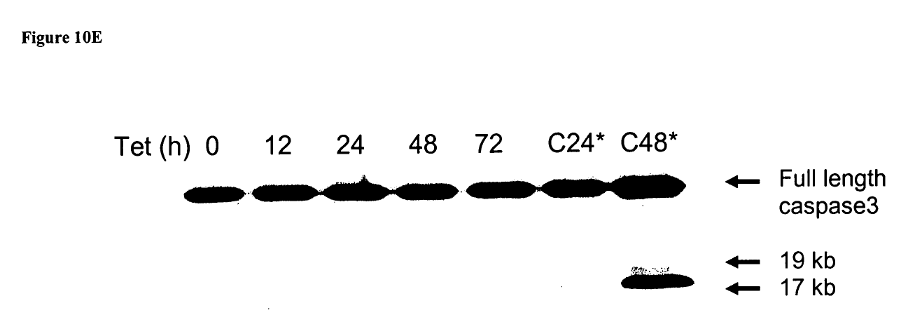

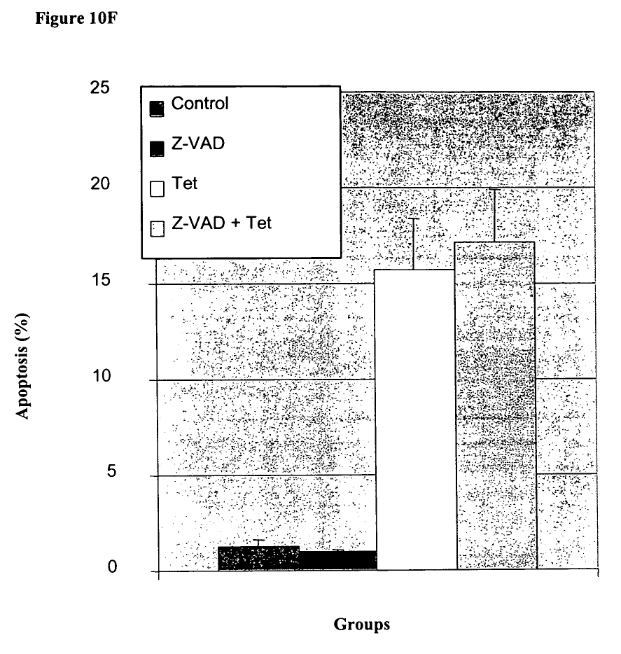

To investigate E2F1 induced apoptosis, an inducible system was established. Human colon cancer cell lines harboring a mutated p53 gene were used to generate E2F1-inducible cell lines (Rodrigues, et al., PNAS, 87:7555-9 (1990)). E2F1 expression was effectively induced and tightly controlled by addition of tetracycline (FIG. 10A). Under the tetracycline induction conditions, a significant percentage of DLD1 cells undergoing apoptosis were detected at a given time point (10% at day 3; 15% at day 4) as determined by propidium iodide (PI) staining and flow cytometry (FIG. 10B). The apoptosis was further confirmed by assaying morphological changes following expression of E2F1 (FIG. 10C). Similar data was obtained in E2F1-inducible SW480 human colon cancer cells. In addition to proapoptotic function, E2F1 is a necessary transcription factor for cell proliferation (Johnson, D. et al., Nature 365:349-52 (1993); Wu, L. et al. Nature 414:457-62 (2001)). Under these experimental conditions, no significant changes in overall cell cycle distribution were observed. Colony formation of cancer cells was completely ablated by E2F1 induction (FIG. 10D), which is consistent with the proapoptotic and tumor suppressor function of E2F1 (Yamasaki, L. et al., Cell 85:537-48 (1996); Field, S. J. et al., Cell 85:549-61 (1996)), suggesting that E2F1 induced apoptosis is different from proapoptotic activity of an oncogene.

### Example 7

### E2F1 Activates Caspase Independent Apoptosis

To further investigate the mechanism of E2F1-induced apoptosis, the role of caspase activation was investigated. Two forms of caspase-3, procaspase and cleaved caspase were detected by western blot analysis. Although the cells clearly underwent apoptosis following expression of E2F1, there was surprisingly no activation of caspase-3, the final effector of caspase-dependent apoptosis following tetracycline induction for 0-72 hours and controls at 24 and 48 hours (FIG. 10E). Further, it was determined if E2F1-induced, p53 independent apoptosis was affected by inhibiting caspase function with a pancaspase inhibitor. Apoptosis induced by camptothecin, a known caspase-3 activator, was used as a positive control. Addition of the pancaspase inhibitor Z-VAD at 50 μM blocked camptothecin-induced apoptosis but failed to block E2F1-induced apoptosis (FIG. 10F). Z-VAD addition alone had no cytotoxic effects. These results suggest that E2F1 induces a caspase-independent cell death pathway in p53 mutant cancer cells.

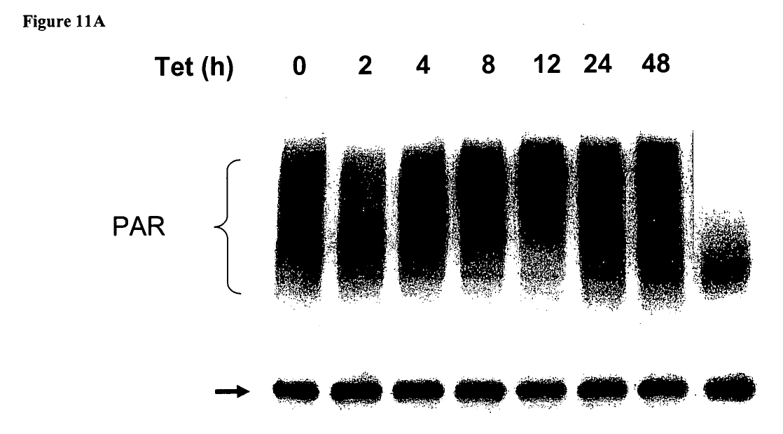

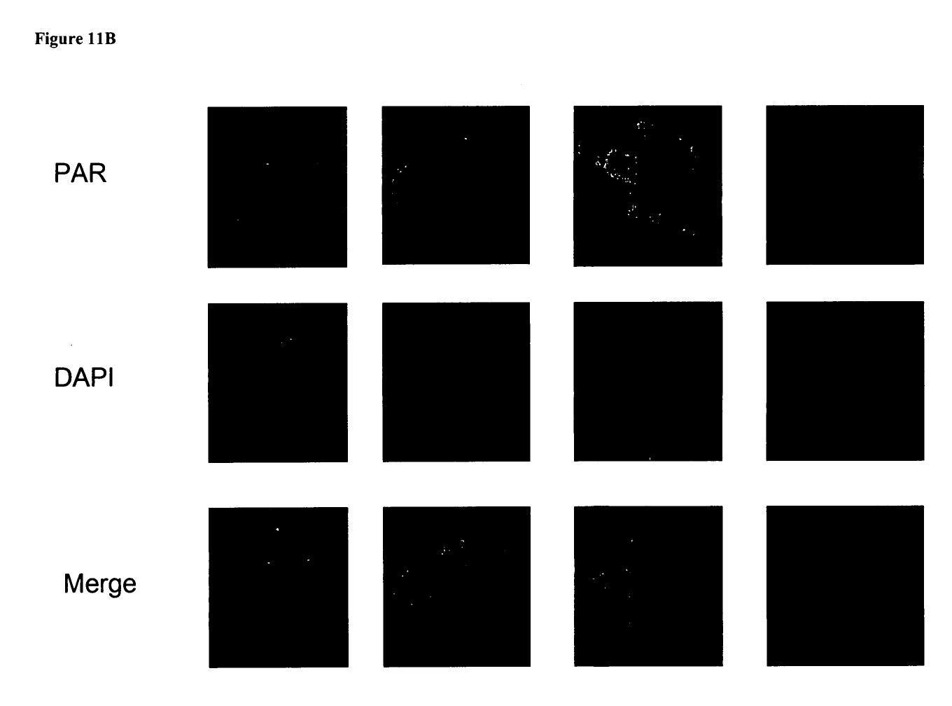

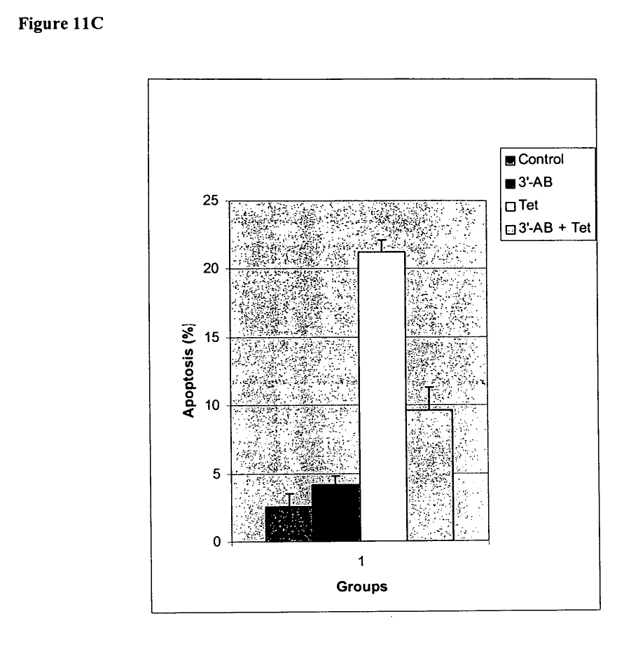

Activation of PARP-1 has been implicated in caspase independent apoptosis. It was investigated whether E2F1 induced cell death involved PARP-1 activation. Poly ADP ribosylation (PAR), the functional indicator of PARP-1 activation, was detected by immunoblot with a specific anti-PAR antibody. As shown in FIG. 11A, E2F1 strongly activated poly ADP ribosylation of proteins. Immunocytochemical staining of PAR further conformed the activity of PARP-1 (FIG. 11B). The nuclear PAR stain was increased after expression of E2F1 for 24 h and reached a plateau at 48 h. To determine if poly ADP ribosylation plays a causal role in E2F1 induced apoptosis, 3′-aminobenzamide (3′-AB), a universal inhibitor of PARP activity, was used. As shown in FIG. 11C, 3′-AB inhibited E2 μl induced apoptosis by more than 70% at the concentration of 5 mM (P<0.0001). 3′-AB addition alone showed no effect on apoptosis over the control group. These data suggest that E2F1 activates protein poly ADP ribosylation that contributes to caspase independent apoptosis.

### Example 8

### E2F Affects Protein Expression of PARP

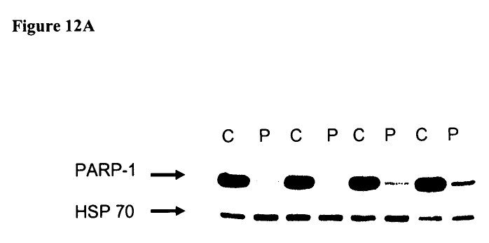

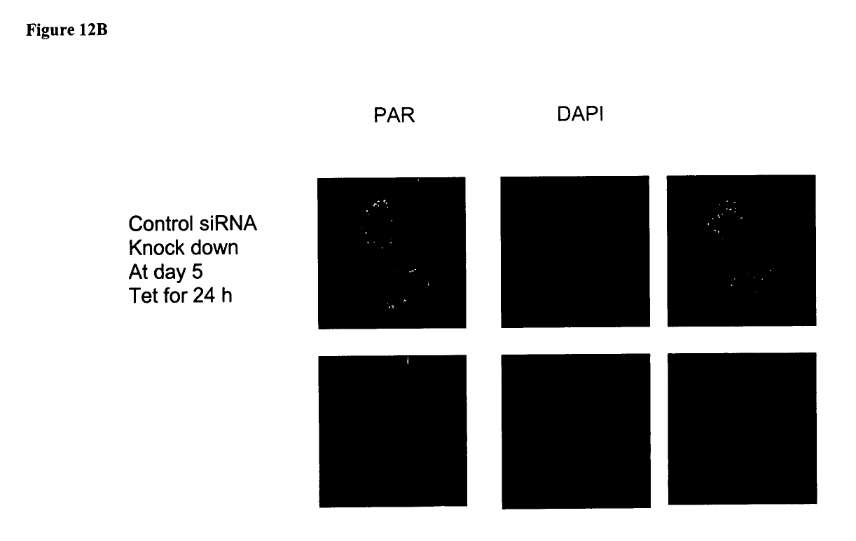

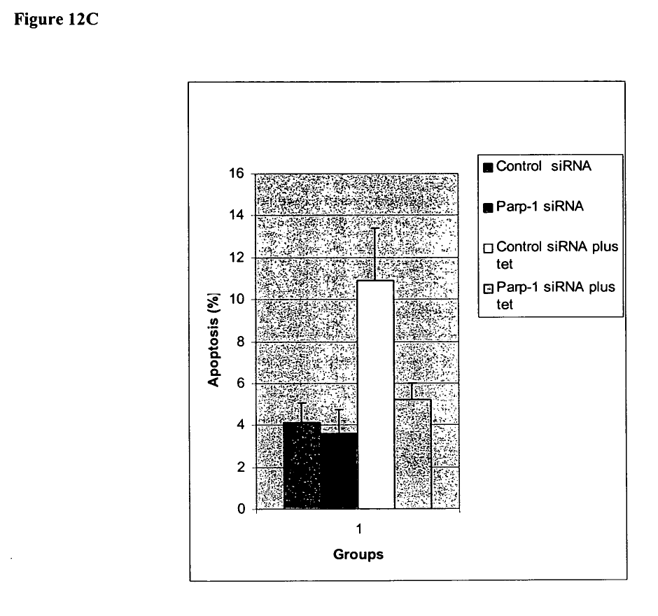

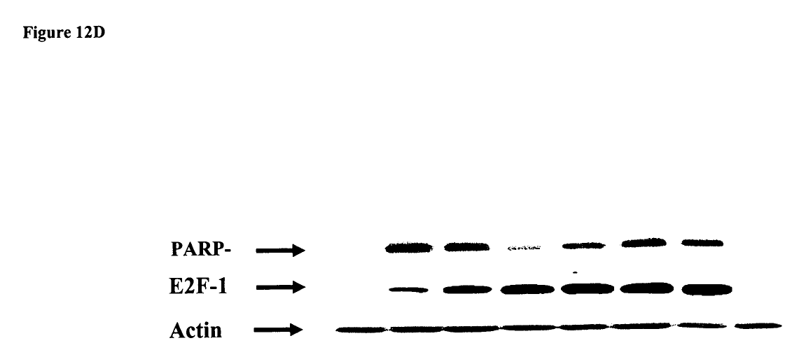

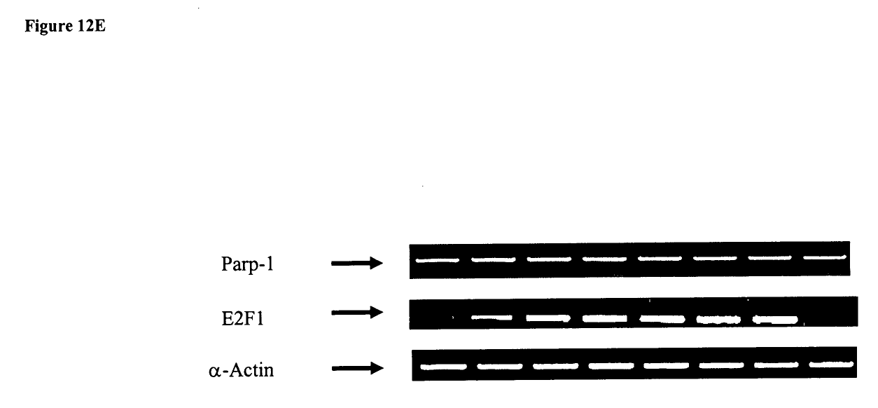

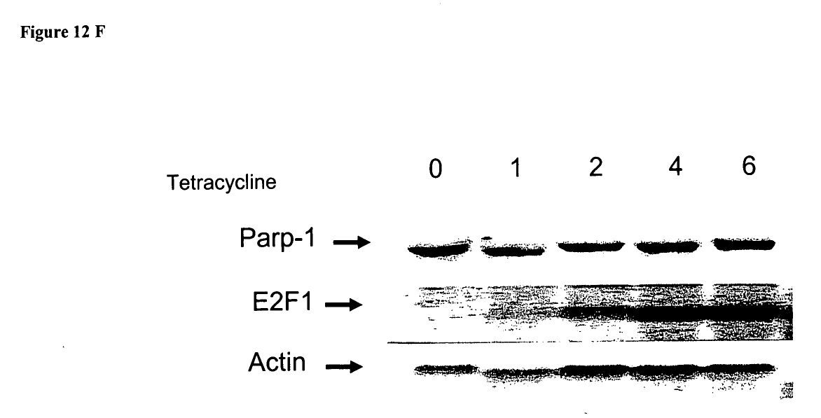

Protein poly ADP ribosylation is catalysed by members of the poly (ADP-ribose) polymerase family that consists of 18 genes (Amé, J. C., et al., Bioessays 26:882-93 (2004)). The PARP-1 protein is the dominant member in most cells, which contributes the majority of overall poly ADP ribosylation induced by DNA damage (Davidovic, L. et al., Exp Cell Res 268:7-13 (2001)). To distinguish which member of the PARP family was involved in E2F1-induced apoptosis, siRNA technology was utilized to reduce PARP-1 protein levels. A chemically synthesized PARP-1 siRNA pool was electroporated into the DLD1-E2F1 inducible cell line. The PARP-1 protein began to decrease at day 2 and reached the lowest levels at day 4 and gradually recovered from day 6 as shown by western blot (FIG. 12A) and immunocytochemical staining. Following expression of E2F1, the nuclear accumulation of PARP was abrogated by transfection of siPARP-1 (FIG. 12B), suggesting that E2F1-induced PARP activity is mediated by PARP-1. The E2F1-induced apoptosis was compromised following PARP-1 knockdown (P<0.001) (FIG. 12C). Taken together, these data suggest that E2F1 requires PARP-1 and its activity to activate a caspase-independent apoptosis pathway.

E2F1 is known as a transcription factor. Yet several lines of evidence suggest that the transcriptional activity of E2F1 is dispensable for its apoptotic function (Phillips, A. C., et al., Genes Dev 11:1853-63 (1997); Hsieh, J. K., et al., Genes Dev 11:1840-52(1997)). It was next determined if E2F1 affected transcription of PARP-1. The expression of E2F1 greatly increased PARP-1 protein levels (FIG. 12D), suggesting a possibility that E2F1 induced PARP-1 expression by transactivation of PARP-1. However, RT-PCR and Northern blot analysis revealed that E2F1-induced PARP-1 protein elevation occurred at the protein level, not at mRNA levels (FIG. 12E, FIG. 12F). These results suggest that E2F1 induces PARP-1 through a non-transcriptional mechanism.

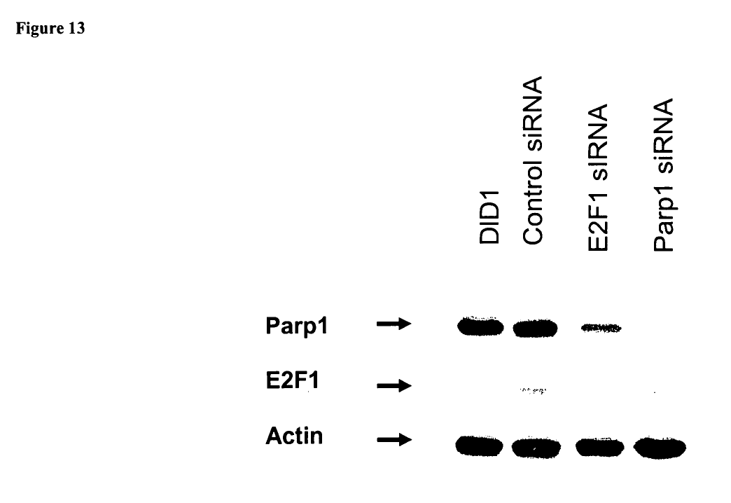

To investigate if endogenous E2F1 affects PARP-1 protein levels, E2F1 was silenced in a non-inducible system. Silencing E2F1 with siRNA reduced endogenous PARP-1 protein level while silencing PARP-1 had no effect on E2F1 protein level (FIG. 13), suggesting a role of E2F1 in regulating PARP-1 protein levels.

### Example 9

### E2F1 Mediates Apoptosis by Inducing Translocation of AIF in a PARP Dependent Manner

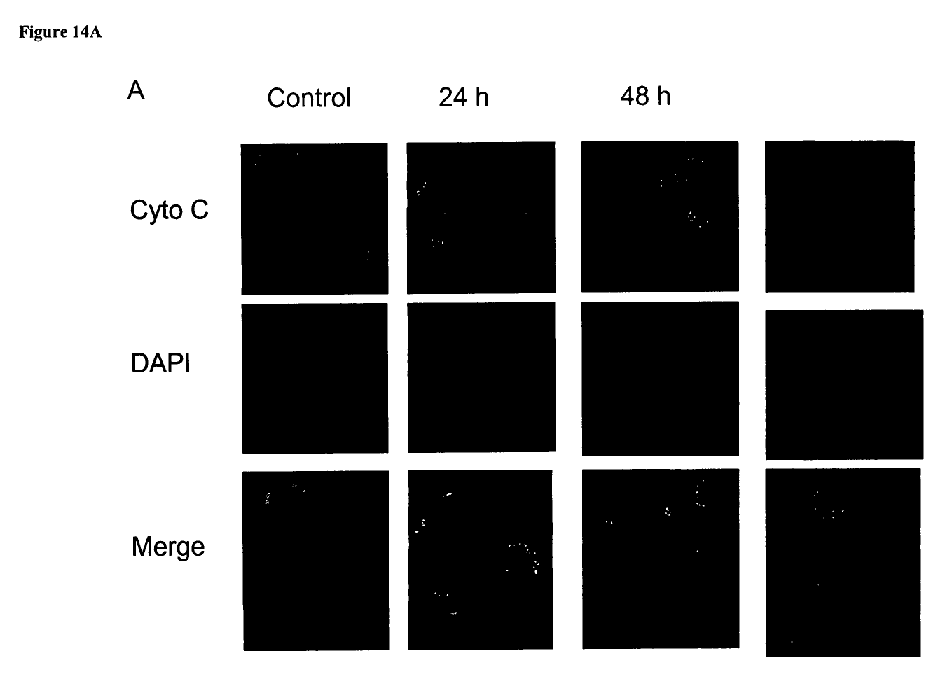

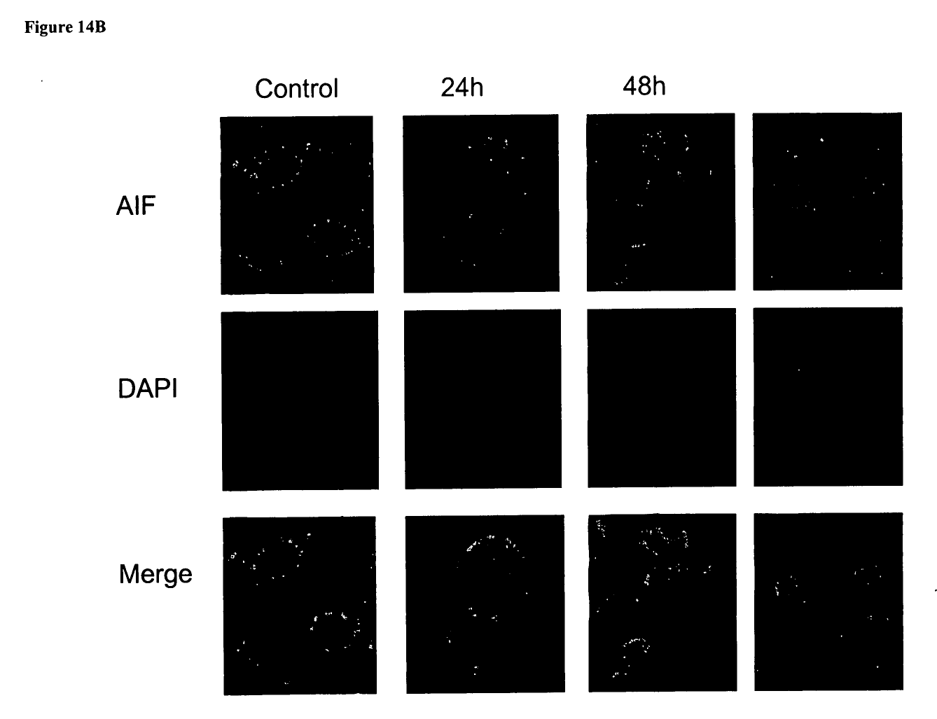

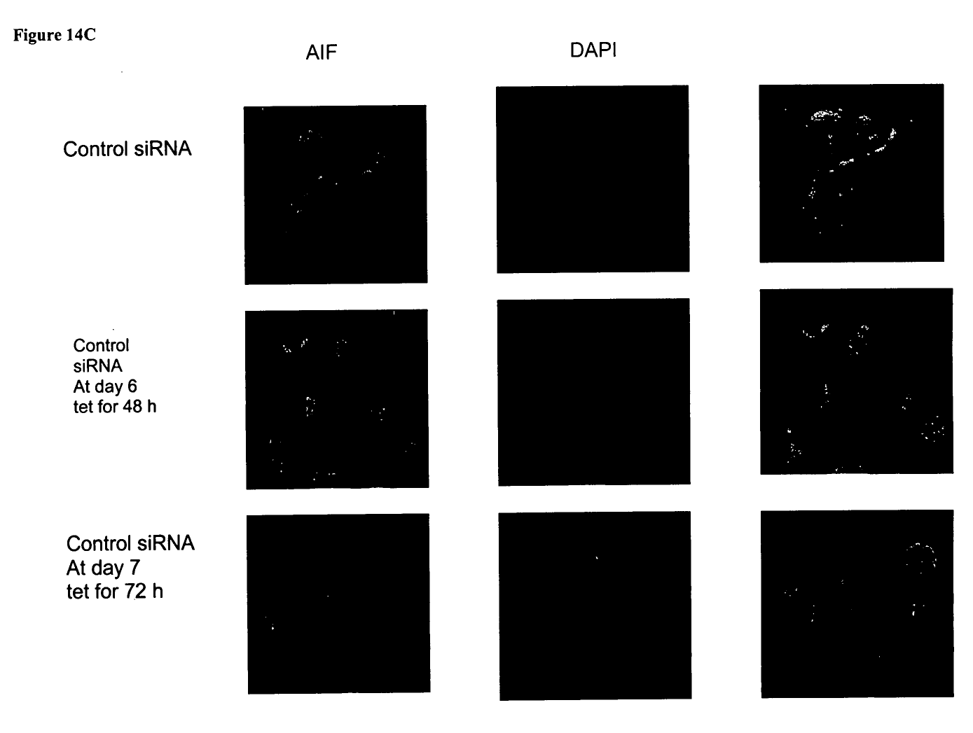

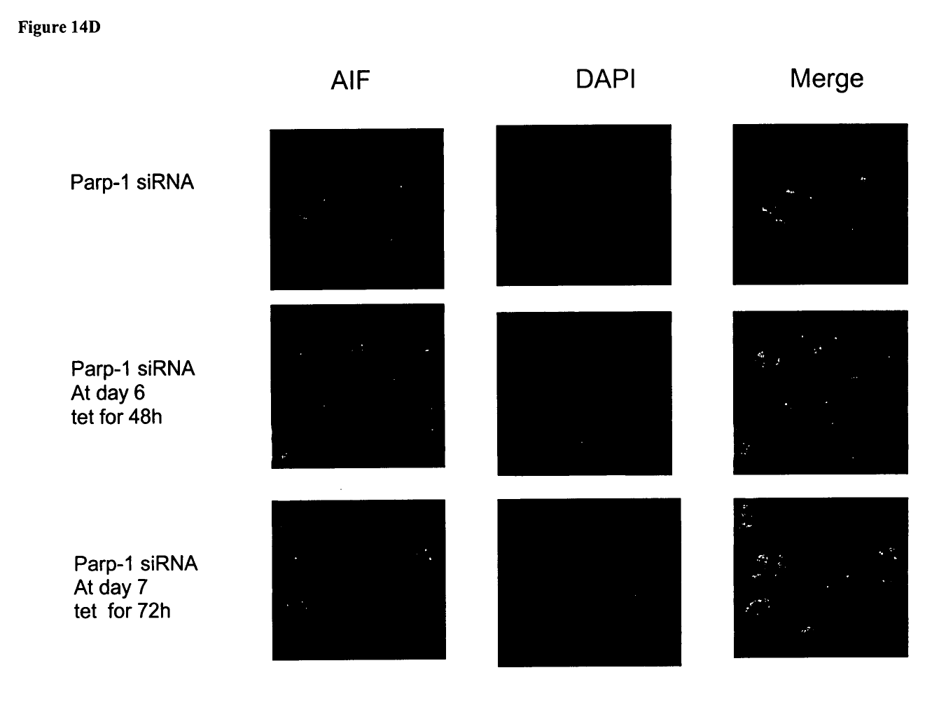

The translocation of apoptosis-inducing factor (AIF) from mitochondria to nuclei has been implicated in the apoptosis induced by PARP-1 (Yu, S. W. et al., Science 297:259-63 (2002); Davidovic, L., et al., Exp Cell Res 268:7-13(2001)). It was next determined if E2F1-mediated increase in PARP-1 protein levels triggered AIF translocation. The expression of E2F1 resulted in AIF and cytochrome c release from mitochondria and translocation of AIF into nuclei. (FIG. 14A, FIG. 14B). To determine if the E2F1 induced AIF translocation was primarily mediated by PARP-1, PARP-1 protein levels were reduced with siRNA. While E2F1 induction drove nuclear translocation of AIF in cells transfected with control siRNA, silencing PARP-1 with a specific siRNA blocked E2F1-induced nuclear translocation of AIF (FIG. 14C, FIG. 14D). These results suggest that the E2F1-PARP-1 apoptosis pathway involves AIF translocation.

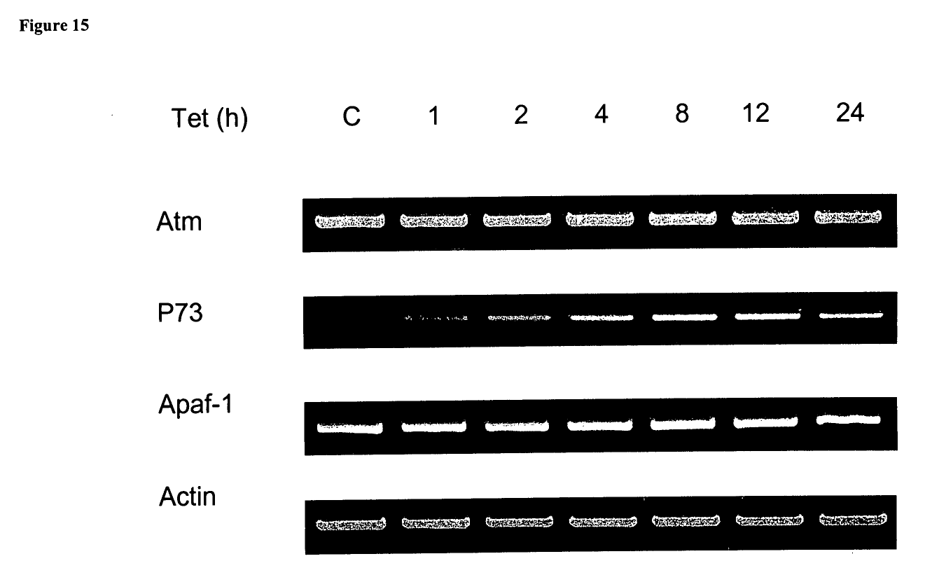

It has been shown that E2F1 transcriptionally regulates apoptotic genes, notably p73 and apaf1, which contribute to transcription-dependent apoptosis pathway of E2F1 (Irwin, M. et al., Nature 407:645-8 (2000); Furukawa, Y. et al., J Biol Chem 277:39760-8 (2002)). p73 was found to be a target gene for E2F1 in the p53 independent apoptosis pathway. Although p73 mRNA was marginally induced after E2F1 expression as detected by RT-PCR (FIG. 15) effective knockdown of p73 by siRNA did not show any protective effect on E2F1-induced apoptosis. E2F1 has also been shown to induce transcription of apaf1 (Furukawa, Y. et al., J Biol Chem 277,39760-8 (2002)). However, RT-PCR data revealed no expression change in apaf1 in the E2F1 inducible cells (FIG. 15). The reason for lack of significant induction of p73 and apaf1 is unclear, and is possibly due to difference in E2F1 levels, duration, and relatively rapid cell death by PARP-1 induction.

In one embodiment, modulation of PARP expression or PARP activity and the resulting modulation of apoptosis occurs via modulation of E2F expression or activity. In a preferred embodiment, E2F is E2F1. The E2F1-PARP-1 cell death pathway provides a p53 independent link between checkpoint regulators and apoptosis. E2F1 is phosphorylated by ATM and Chk2, which leads to the stabilization of E2F1 (Lin, W. C., et al., Genes Dev 15:183344 (2001); Bartek, J., et al., Nat Rev Mol Cell Biol 2:877-86 (2001)). Conversely, E2F1 activates ATM and Chk2 kinase, thereby forming a positive feedback loop (Rogoff, H. A. et al., Mol Cell Biol 24:2968-77 (2004); Berkovich, E. & Ginsberg, D., Oncogene 22:161-7 (2003)). It is speculated that the presence of oncogenic signals or ATM-Chk2 activation due to DNA damage could lead to activation of the E2F1-PARP-1 pathway that may lead to apoptosis, DNA repair or cell cycle arrest depending on the magnitude of the insults, potentially serving an important function in the prevention of tumor formation. This pathway may underlie the observed tumor suppressor function of E2F1 (Yamasaki, L. et al., Cell 85:53748 (1996); Field, S. J. et al, Cell 85:549-61 (1996)).

The interaction between E2F1 and PARP-1 establishes a new link between cell cycle regulation and genome surveillance. Results suggest that the endogenous level of PARP-1 protein is dependent on E2 μl. It has been suggested that PARP-1 enhances transcription activity of E2F1 (Simbulan-Rosenthal, C. M., et al., Oncogene 18:5015-23 (1999); Simbulan-Rosenthal, C. M. et al., Oncogene 22:8460-71 (2003)). These observations suggest a positive feedback loop between genome surveillance and cell cycle regulation. These feedback interactions may offer a highly sensitive mechanism to prevent proliferation of, and to promote elimination of cells with irreparable DNA damage.

The lack of mutations in E2F1 and PARP-1 in human cancers suggests significance of this checkpoint-apoptotic pathway in mediating the anti-cancer activity of chemotherapy, offering potentials for activating this checkpoint pathway to develop efficacious novel therapies against cancers, including those bearing mutations in p53 pathways.

Other embodiments are within the following claims. While several embodiments have been shown and described, various modifications may be made without departing from the spirit and scope of the present invention.

What is claimed is:

1. A method for screening for a PARP activator comprising the step of assessing the PARP-activating effect of a test compound in cells containing DNA encoding PARP.

2. The method of claim 1 wherein the PARP is PARP-1, PARP-2, or both PARP-1 and PARP-2.

3. The method of claim 1 wherein the step of assessing the PARP-activating effect in cells comprising,
exposing the cells to a test compound,
measuring the activity of PARP in the cells in the presence and in the absence of the test compound, and
comparing the activity of PARP in the presence and in the absence of the test compound.

4. The method of claim 1 wherein PARP-activating effect is determined by an increase in poly(ADP ribose) synthesis.

5. The method of claim 1 wherein the cells are cancer cells.

6. The method of claim 5 wherein the cancer cells are cells in a cancer of a vertebrate, mammal, or human.

7. The method of claim 5 wherein the cancer cells are derived from a cancer of a vertebrate, mammal, or human.

8. The method of claim 5 wherein the cancer cells are cultured cancer cells.

9. The method of claim 8 wherein the cultured cancer cells are selected from the group consisting of MCF-7 (human breast cancer cells), DLD1 (human colonic cells), SW480 (human colonic cells), and Paca-2 (human pancreatic cancer cells).

10. The method of claim 1 wherein the test compound is a small molecule.

11. The method of claim 1 wherein the test compound is an analog, derivative, or metabolite of β-lapachone.

12. The method of claim 8 further comprising the step of assessing the PARP-activating effect of the test compound in normal cells containing DNA encoding PARP.

13. The method of claim 12 wherein the step of assessing the PARP-activating effect in normal cells comprising,
exposing the normal cells to a test compound,
measuring the activity of PARP in the normal cells in the presence and in the absence of the test compound, and
comparing the activity of PARP in the presence and in the absence of the test compound.

14. The method of claim 12 wherein the normal cells are normal cells in a vertebrate, mammal, or human.

15. The method of claim 12 wherein the normal cells are normal cells derived from a vertebrate, mammal, or human.

16. The method of claim 12 wherein the normal cells are cultured normal cells.

17. The method of claim 16 wherein the cultured normal cells are selected from the group consisting of MCF-10A (nontransformed breast epithelial cells), NCM460 (normal colonic epithelial cells), PBMC (proliferating peripheral blood mononuclear cells)

18. The method of claim 12 further comprising the step of selecting the test compound that has a higher PARP-activating effect in the cancer cells than in the normal cells.

19. A method for screening for a PARP activator, comprising the step of assessing the PARP-activating effect of a test compound in the lysate of cells containing DNA encoding PARP.

20. The method of claim 19 wherein the cells are cancer cells.

21. The method of claim 20 further comprising,
assessing the PARP-activating effect of the test compound in the lysate of normal cells containing DNA encoding PARP, and
comparing the PARP-activating effects of the test compound in the cancer cell lysate and the normal cell lysate.

22. A method for screening for a PARP activator comprising,
contacting PARP with a test compound,
measuring the activity of PARP in the presence and in the absence of the test compound, and
comparing the activity of PARP in the presence and in the absence of the test compound.

23. The method of claim 22 wherein the PARP is PARP-1 or PARP-2.

24. The method of claim 22 further comprising selecting the test compound that increases the PARP activity.

25. The method of claim 24 further comprising,
assessing the PARP-activating effect of the selected compound in cancer cells containing DNA encoding PARP, or the lysate of the cells,
assessing the PARP-activating effect of the selected compound in the lysate of normal cells containing DNA encoding PARP, or the lysate, and
comparing the PARP-activating effects of the selected compound in the cancer cells or the lysate and the normal cells or the lysate.

26. A method of treating or preventing cancer in a subject comprising increasing PARP activity in cancer cells of the subject.

27. The method of claim 26 comprising selectively increasing PARP activity in cancer cells of the subject.

28. The method of claim 26 comprising administering to the subject a therapeutically effective amount of a PARP activator.

29. The method of claim 26 comprising administering to the subject a therapeutically effective amount of a selective activator of PARP.

30. The method of claim 29 wherein the compound is an analog, derivative, or metabolite of β-lapachone.

31. The method of claim 26, wherein the subject is a vertebrate, mammal, or human.
